(19) United States
(12) Patent Application Publication
(10) Pub. No.: US 2025/0064080 A1
(43) Pub. Date: Feb. 27, 2025

(54) Dairy-Like Compositions

(71) Applicant: Change Foods, Inc., Palo Alto, CA (US)

(72) Inventors: Valentino Setoa Junior Te’o, San Jose, CA (US); David Bucca, San Jose, CA (US)

(21) Appl. No.: 18/726,788

(22) PCT Filed: Jan. 4, 2023
(86) PCT No.: PCT/US2023/060108

§ 371 Date: Jul. 5, 2024

Related U.S. Application Data
(60) Provisional application No. 63/296,790, filed on Jan. 5, 2022.

(51) Int. Cl. A23J 1/00; A23C 11/10; A23C 20/02; A23J 3/20

(52) U.S. Cl. CPC A23J 1/008; A23C 11/10; A23C 20/02; A23J 3/20

(57) ABSTRACT

Compositions comprising one or more recombinant cascin proteins or fragments of recombinant cascin proteins and microbial lipids and methods of producing such compositions are provided.

### CROSS-REFERENCE TO RELATED APPLICATIONS

This application claims the benefit of U.S. Provisional Application No. 63/296,790, filed Jan. 5, 2022, which is hereby incorporated by reference in its entirety.

### SEQUENCE LISTING

The instant application contains a Sequence Listing which has been submitted via Patent Center and is hereby incorporated by reference in its entirety. Said XML copy, created on Dec. 30, 2022, is named 53962WO_sequencelisting.xml, and is 22,514 bytes in size.

### FIELD OF THE DISCLOSURE

The present disclosure relates to dairy-like compositions and in particular to compositions comprising recombinant dairy proteins and microbial lipids.

### BACKGROUND OF THE DISCLOSURE

Any discussion of the prior art throughout the specification should in no way be considered as an admission that such prior art is widely known or forms part of the common general knowledge in the field.

Modern agriculture is substantially more productive now than it was only a decade ago. Despite this, agriculture still consumes vast quantities of resources and is a major contributor to greenhouse gas emissions, human sickness, environmental damage and animal suffering. The threats posed by agriculture will only become greater as global dairy consumption increases with human population growth.

Agriculture has been reported to use more freshwater than any other human activity, with nearly a third required for livestock. About 98% of the water used in livestock production goes to animal feed (Godfray et al., 2018, Science. 361:243).

According to the Food and Agriculture Organisation of the United Nations, about 18% of all greenhouse gas emissions are a consequence of animal agriculture. Indeed, animal agriculture contributes more to greenhouse gas emissions than the entire transportation sector. Livestock alone accounts for about 5% of the CO₂ that humans add to the atmosphere. Amplifying the consequence of these emissions is the loss of natural habitats that might otherwise sequester carbon emissions. The majority of tropical deforestation is for the purpose of feeding animals.

Animal agriculture also presents health and safety risks. Livestock can act as a reservoir for pathogens that infect humans, including Salmonella and Campylobacter. Intensive factory farming is thought to be responsible for foodborne illnesses such as swine influenza and avian influenza. Moreover, the widespread use of antibiotics in animal agriculture can promote the development of antibiotic-resistant pathogens.

As well as its impact on the environment, animal agriculture can have adverse consequences on animal welfare. This is the case whether the animals are farmed for meat or milk production. Animal milk and milk products also comprise components that can have unhealthy consequences in humans (eg, lactose, allergens, cholesterol), and they may be prone to microbial contamination.

In this context, there is a need for alternative sources of dairy products. It is an object of the present invention to overcome or ameliorate at least one of the disadvantages of the prior art, or to provide a useful alternative.

### SUMMARY OF THE DISCLOSURE

In work leading to the present disclosure, the inventors found that milk proteins such as caseins can be recombinantly expressed, purified at high yields and combined with yeast lipids to form a dairy-like composition, without any animal-derived milk. The composition forms a curd structure which is stable at room temperature. It is dairy-like in that it resembles an animal-derived dairy product such as milk, cheese or curd, comprising a matrix of caseins and fats. The compositions described herein may be used in the manufacture of food and drink products with little or no milk.

In one aspect, the present disclosure provides a dairy-like composition comprising: one or more recombinant casein proteins or fragments thereof; and microbial lipids. The compositions described herein may be formed in an aqueous solution such as water.

The composition is preferably milk-free.

In some examples, the one or more recombinant casein proteins are selected from the group consisting of αS1-casein, αS2-casein, β-casein and κ-casein. In some examples, the one or more casein proteins comprise αS1-casein and/or β-casein. At least one of the recombinant casein proteins may be expressed in a microorganism from a codon optimised gene. In some examples, the one or more casein proteins are produced by a microorganism. In some examples, the one or more casein proteins and the microbial lipids are produced by different microorganisms. In some examples, the one or more casein proteins and the microbial lipids are produced by different species of microorganisms. The recombinant casein proteins may be isolated from E. coli or a species of Trichoderma.

In some examples, the recombinant casein proteins is isolated from a microorganism by a process comprising: i) lysing the microorganism to produce a cell lysate; ii) heating the cell lysate to produce a heat-treated lysate; iii) centrifuging the heat-treated lysate and obtaining a supernatant; iv) adding an acid to the supernatant so as to lower its pH and promote precipitation of the casein protein; and v) centrifuging the supernatant to form a pellet of casein protein. The process may further comprise: vi) resuspending the pellet of casein protein in an aqueous solution; and vii) freeze drying or spray drying the resuspended casein protein.

In some examples, the microbial lipids are produced by a microorganism that is not recombinant. In some examples, the microbial lipids are yeast lipids. The yeast may be Debaryomyces hansenii, Kluyveromyces lactis or Yarrowia lipolytica. In some examples, the yeast is Debaryomyces hansenii.

In some examples, the composition comprises whole yeast cells, and wherein the yeast cells comprise the lipids. The yeast cells may be obtained by spray drying or freeze drying. In some examples, the composition comprises lysed spray dried yeast cells, and wherein the yeast cells comprise the lipids. In some examples, the composition comprises lipids extracted from yeast cells.

In some examples, the composition comprises recombinant casein proteins at a concentration of between 5% and 25% by weight.

In some examples, the composition comprises microbial lipids at a concentration of between 1% and 20% by weight.

In some examples, the composition further comprises a plant oil or a plant protein.

In some examples, the composition comprises microbial lipids at a concentration of between 1% and 5% by weight. In some examples, the microbial lipids comprise myristic acid, palmitic acid, stearic acid, oleic acid, linoleic acid and linolenic acid. In some examples, the composition comprises between about 4% and 40% by weight lipids, and wherein a fraction of the lipids are microbial lipids. In some examples, the composition comprises between about 1% and 5% by weight microbial lipids and between about 5% and 40% by weight total lipids.

In another aspect, the present disclosure provides a method of producing a dairy-like composition the method comprising mixing one or more recombinant casein proteins or fragments thereof with microbial lipids. In some examples, the one or more recombinant casein proteins are selected from the group consisting of αS1-casein, αS2-casein, β-casein and κ-casein. In some examples, the one or more casein proteins comprise αS1-casein and/or β-casein. At least one of the recombinant casein proteins may be expressed in a microorganism from a codon optimised gene. In some examples, the one or more casein proteins are produced by a microorganism. In some examples, the one or more casein proteins and the microbial lipids are produced by different microorganisms. In some examples, the one or more casein proteins and the microbial lipids are produced by different species of microorganisms. The recombinant casein proteins may be isolated from E. coli or a species of Trichoderma.

The composition is preferably milk-free.

In some examples, the mixing is performed at a temperature of between about 70° C. and 80° C.

In some examples, the microbial lipids are yeast lipids. The yeast may be Debaryomyces hansenii, Kluyveromyces lactis or Yarrowia lipolytica. In some examples, the lipids are added to the composition in the form of whole yeast cells comprising the lipids. In some examples, the lipids are added to the composition in the form of lysed yeast cells comprising the lipids. The yeast cells may be prepared by spray drying or freeze drying. In some examples, the lipids are extracted from yeast cells. In some examples, the one or more recombinant casein proteins are isolated from a microorganism. In some examples, the one or more recombinant casein proteins are isolated from bacteria. In some examples, the one or more recombinant casein proteins are isolated from E. coli or Trichoderma.

In another aspect, the present disclosure provides a food or beverage product comprising a dairy-like composition as described herein.

### BRIEF DESCRIPTION OF THE DRAWINGS

FIG. 1. Fermentation batch run of E. coli expressing recombinant casein.

FIG. 2. Downstream processing of E. coli expressing casein proteins. Fermentation (A). E.coli cells (B). Wet cell paste (C). Cell lysate (D). Cell lysate following heat treatment (E). Supernatant (F). SDS-PAGE of supernatant; arrows indicate caseins (G). Acid-induced (pl) precipitation (H). Wet casein aggregate following acid-induced precipitation (I). Spray drying (J). Casein powder (K).

FIG. 3. Agarose gel electrophoresis following 2-stage purification.

FIG. 4. Carbon to nitrogen (C:N) testing for lipid production in D. hansenii (A), K. lactis (B) and Y. lipolytica (C).

FIG. 5. Microscopy images of D. hansenii CF 10 unstained (left) and stained (right).

FIG. 6. Fed-batch fermentation of yeast.

FIG. 7. Spray dried yeast.

FIG. 8. Lipid production by D. hansenii and K. lactis. Cell mass (left bar) and lipid content (right bar).

FIG. 9. Recombinant casein proteins mixed with water before coagulation (A) and after coagulation (B).

FIG. 10. Dairy-like compositions comprising extracted yeast lipids. Components alpha S1, beta casein and yeast oil shown in (A). Mixture of alpha S1, beta casein and yeast oil shown in (B) and the formation of a curd-like matrix of alpha S1, beta casein and yeast oil shown in (C).

FIG. 11. Dairy-like compositions comprising whole yeast. Components alpha S1, beta casein and whole yeast shown in (A). Mixture of alpha S1, beta casein and whole yeast shown in (B) and the formation of a curd-like matrix of alpha S1, beta casein and whole yeast shown in (C).

FIG. 12. Scanning electron microscopy image of a dairy-like composition comprising alpha S1, beta casein and yeast oil. Arrows indicate yeast oil droplets within aggregated casein proteins forming a curd-like matrix.

FIG. 13. Scanning electron microscopy image of a dairy-like composition comprising alpha S1, beta casein and whole yeast. Arrows indicate yeast cells within aggregated casein proteins forming a curd-like matrix.

FIG. 14. Dairy-like composition comprising lysed spray dried yeast. Recombinant alpha S1 is shown in (A). Lysed spray dried yeast is shown in (B). The alpha S1 and lysed spray dried yeast were mixed in water (C) and heated to produce a curd-like matrix (D).

FIG. 15. Dairy-like composition comprising lysed spray dried yeast. Recombinant alpha S1 is shown in (A). Lysed spray dried yeast is shown in (B). Sunflower oil is shown in (C). The components were mixed in water (D) and heated to produce a curd-like matrix (E).

FIG. 16. Dairy-like composition comprising recombinant caseins. Components recombinant alpha S1 casein, beta casein and sunflower oil are shown in (A). The components were mixed in water (B) and heated to produce a curd-like matrix (C).

FIG. 17. Composition comprising dairy caseins. Rennet casein is shown in (A). Sunflower oil is shown in (B). The components were mixed in water (C) and heated (D).

FIG. 18. Scanning electron microscopy image of a dairy-like composition comprising recombinant alpha S1, recombinant beta casein and lysed spray dried yeast.

FIG. 19. Scanning electron microscopy image of a dairy-like composition comprising recombinant alpha S1, pea protein, sunflower oil and lysed spray dried yeast. Arrows indicate oil droplets.

FIG. 20. Recombinant alpha S1 cheese composition stretching following cooking and cooling down.

FIG. 21. Output data showing viscosity (cP), temperature (° C.), and agitation speed (rpm) trends during recombinant alpha S1 casein cheese manufacture. Referring to time point 12 min, the top line corresponds to speed (rpm), the middle line corresponds to temperature (° C.) and the bottom line corresponds to viscosity.

FIG. 22. Confocal images (40×) of cheese samples stained with dyes Nile Red (for lipids droplets) and Fast Green FCF (for proteins). Top contains recombinant alpha S1 casein. Bottom contains dairy Rennet caseins (composed of Alpha S1, Alpha S2 and Beta caseins). Dark areas show gaps.

### DETAILED DESCRIPTION

### Definitions

In the context of this specification, the terms “a” and “an” are used herein to refer to one or to more than one (i.e. to at least one) of the grammatical object of the article. By way of example, “an element” means one element or more than one element.

The term “about” is understood to refer to a range of +/−10%, preferably +/−5% or +/−1% or, more preferably, +/−0.1%.

The terms “comprise”, “comprises”, “comprised” or “comprising”, “including” or “having” and the like in the present specification and claims are used in an inclusive sense, ie, to specify the presence of the stated features but not preclude the presence of additional or further features.

The term “fat” typically refers to a lipid composition that is solid at ambient conditions (ie, 20° C.-30°° C. and 0.95-1.05 atm).

The term “fatty acid profile” as used herein refers to the distribution of fatty acids (eg, distribution of types of fatty acids and/or abundances of distinct types of fatty acids and/or relative amounts of distinct types of fatty acids) in a composition without reference to attachment to a glycerol backbone or reference to the regiospecific nature of any connection to a glycerol backbone. Fatty acid profiles are typically determined by conversion to a fatty acid methyl ester (FAME), followed by gas chromatography (GC) analysis with flame ionization detection (FID). A fatty acid profile can be expressed as percent of a fatty acid in a total fatty acid signal determined from the area under the curve for that fatty acid.

The term “free fatty acid” as used herein refers to a fatty acid that is not bound to a glycerol backbone.

The term “isolated” as used herein refers to material that is substantially or essentially free from components that normally accompany it in its native state. For example, an isolated polynucleotide refers to a polynucleotide which has been purified from the sequences which flank it in a naturally-occurring state, eg, a DNA fragment which has been removed from the sequences that are normally adjacent to the fragment.

The term “lipid” as used herein includes an organic compound that is soluble in nonpolar solvents (such as ether and chloroform) and is relatively or completely insoluble in water. Non-limiting examples of lipids include glycerolipids (eg, monoglycerides, diglycerides, triglycerides, neutral fats, phosphoglycerides, glycerophospholipids), nonglycerides (eg, sphingolipids, sterol lipids [e.g., cholesterol, steroid hormones), prenollipids [eg, terpenoids], fatty alcohols, fatty acids, waxes, polyketides), and complex lipid derivatives (eg, sugar-linked lipids, glycolipids, protein-linked lipids). Lipids natively present in animal milk may include milk triglyceride (mTAG), milk diglyceride (mDAG), milk monoglyceride (mMAG), milk phospholipid (mPL), milk free fatty acid (mFFA) and milk sterol.

A “milk-free” composition is a composition comprising no animal milk. A milk-free composition is essentially free of components obtained from animal milk. The term “essentially free of” as used herein refers to the indicated component being either not detectable in the composition by common analytical methods, or being present in such trace amount as to not be functional. The term “functional” as used in this context refers to not contributing to properties of the composition comprising the trace amount of the indicated component, or to not having activity (e.g., chemical activity, enzymatic activity) in the indicated composition comprising the trace amount of the indicated component, or to not having health-adverse effects upon use or consumption of the composition comprising the trace amount of the indicated component.

The term “oil” typically refers to a lipid composition that is liquid at ambient conditions (i.e., 20° C.-30° C. and 0.95-1.05 atm).

The term “recombinant cell” as used herein refers to a cell that comprises a recombinant polynucleotide. Thus, for example, a recombinant cell may produce a polynucleotide or polypeptide not found in the native (non-recombinant) form of the cell, or a recombinant cell may produce a polynucleotide or polypeptide at a level that is different from that in the native (non-recombinant) form of the host cell. It should be understood that such term is intended to refer not only to the particular subject cell but also to the progeny of such a cell.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

The terms “recombinant polypeptide” and “recombinant protein” as used herein refer to a polypeptide made using recombinant techniques, ie, through the expression of a recombinant polynucleotide. A fragment of a recombant protein may be produced by expression of a recombinant polynucleotide encoding the protein fragment, or by fragmentation (eg, enzymatic or chemical digestion) of a full-length recombinant protein.

Where numerical ranges are used to describe certain embodiments of the present disclosure, it will be understood that each range should be considered to encompass subranges therein. For example, the description of a range such as from 1 to 6 should be considered to include subranges such as from 1 to 5, from 2 to 4, from 2 to 6 and so on. Likewise, the description of a range of between 1 and 6 should be considered to include subranges such as between 2 and 5, between 1 and 3, between 3 and 6 and so on.

### Recombinant Milk Proteins

Dairy-like compositions of the present disclosure preferably comprise milk proteins such as caseins that are recombinantly expressed in a microorganism. Suitable casein proteins may include any one or more of αS1-casein, αS2-casein, β-casein, γ-casein and κ-casein. The composition of the present disclosure may comprise a recombinant casein protein selected from the group consisting of αS1-casein, αS2-casein, β-casein, γ-casein and κ-casein.

In some examples, the composition comprises two recombinant casein proteins, such as: αS1-casein and αS2-casein; αS1-casein and β-casein; αS1-casein and γ-casein; αS1-casein and κ-casein; αS2-casein and β-casein; αS2-casein and γ-casein; αS2-casein and κ-casein; β-casein and γ-casein; β-casein and κ-casein; or γ-casein and κ-casein. In some examples, the composition comprises three recombinant casein proteins, such as: αS1-casein, αS2-casein and β-casein; αS1-casein, αS2-casein and γ-casein; αS1-casein, αS2-casein and κ-casein; αS2-casein, β-casein and γ-casein; αS2-casein, β-casein and κ-casein; αS2-casein, γ-casein and κ-casein; β-casein, γ-casein and κ-casein; αS1-casein, β-casein and γ-casein; αS1-casein, β-casein and κ-casein; or αS1-casein, γ-casein and κ-casein. In some examples, the composition comprises four recombinant casein proteins, such as: αS1-casein, αS2-casein, β-casein and γ-casein; αS1-casein, αS2-casein, β-casein and κ-casein; αS1-casein, β-casein, γ-casein and κ-casein; αS1-casein, αS2-casein, γ-casein and κ-casein; or αS2-casein, β-casein, γ-casein and κ-casein.

In some examples, the composition comprises recombinant αS1-casein, αS2-casein, β-casein, γ-casein and κ-casein. The recombinant caseins may be comprised of between about 33% and 43% αS1-casein, between about 5% and 15% αS2-casein, between about 31% and 41% β-casein, between about 7% and 18% κ-casein, and between about 1% and 8% γ-casein. In some examples, the recombinant caseins are comprised of between about 35% and 41% αS1-casein, between about 7% and 13% αS2-casein, between about 33% and 39% β-casein, between about 9% and 16% κ-casein, and between about 1% and 6% γ-casein. In some examples, the recombinant caseins are comprised of between about 36% and 40% αS1-casein, between about 8% and 12% αS2-casein, between about 34% and 38% β-casein, between about 10% and 15% κ-casein, and between about 2% and 5% γ-casein.

Recombinant casein proteins may be present in the composition at a concentration of between about 1% and 50%, such as between about 1% and 25%, or between about 1% and 20%, or between about 2% and 20%, or between about 3% and 20%, or between about 4% and 20%, or between about 5% and 20%, or between about 10% and 20%, or between about 8% and 18%, or between about 15% and 20%. In some examples, recombinant casein may be present in the composition at a concentration of between about 1% and 5%, or between about 2% and 5%, or between about 3% and 5%. In some examples, recombinant casein may be present in the composition at a concentration of between about 1% and 30%, such as between about 2% and 20% or between about 5% and 20%, or between about 5% and 15%. It will be understood that the present disclosure enables compositions to be made having desired protein concentrations, including desired casein concentrations, as well as desired protein (including casein) profiles.

In some examples, the composition comprises between about 1% and 20% αS1-casein, such as between about 2% and 20%, or between about 3% and 20%, or between about 4% and 20%, or between about 5% and 20%, or between about 5% and 15%, or between about 5% and 10%, or between about 1% and 5% αS1-casein. In some examples, the composition comprises between about 1% and 20% αS2-casein, such as between about 2% and 20%, or between about 3% and 20%, or between about 4% and 20%, or between about 5% and 20%, or between about 5% and 15%, or between about 5% and 10%, or between about 1% and 5% αS2-casein. In some examples, the composition comprises between about 1% and 20% beta-casein, such as between about 2% and 20%, or between about 3% and 20%, or between about 4% and 20%, or between about 5% and 20%, or between about 5% and 15%, or between about 5% and 10%, or between about 1% and 5% beta-casein. In some examples, the composition comprises between about 1% and 20% kappa-casein, such as between about 2% and 20%, or between about 3% and 20%, or between about 4% and 20%, or between about 5% and 20%, or between about 5% and 15%, or between about 5% and 10%, or between about 1% and 5% kappa-casein. In some examples, αS1-casein and beta-casein are the most abundant casein proteins in the composition. For example, αS1-casein and beta-casein may comprise at least 50%, such as at least 60%, or at least 70% or at least 80%, or at least 90% of the casein proteins present in the composition. In some examples, αS1-casein, beta-casein and αS2-casein comprise at least 75%, such as at least 80%, or at least 85%, or at least 90%, or at least 95%, or at least 99% of the casein proteins present in the composition. In some examples, αS1-casein comprises between about 40% and 50% of the casein proteins present in the composition, beta-casein comprises between about 40% and 50% of the casein proteins present in the composition and αS2-casein comprises between about 5% and 15% of the casein proteins present in the composition. In some examples, αS1-casein comprises about 45% of the casein proteins present in the composition, beta-casein comprises about 45% of the casein proteins present in the composition and αS2-casein comprises about 10% of the casein proteins present in the composition.

The amino acid sequence of the casein may be the same as, or similar to, that found in an animal such as a mammal. For example, the amino acid sequence of the casein may be the same as, or similar to, that found in cow, human, sheep, goat, gorilla, elephant, wallaby, kangaroo, whale, possum, tiger, lion, buffalo, lama, bison, horse or camel. In some examples, the nucleotide sequence encoding the casein protein is codon optimised for expression in a host microorganism.

The casein protein may have a glycosylation or phosphorylation pattern which is the same as, similar to, or different from, that which is found in animal-derived caseins. In some examples, the casein protein has no post-translational modifications.

In some examples, the present disclosure provides a composition comprising recombinant αS1-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 1 or a fragment thereof. In some examples, the present disclosure provides a composition comprising recombinant αS1-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 2 or a fragment thereof. It will be understood, however, that any αS1-casein may be suitable for inclusion in a composition of the present disclosure, and that the caseins described herein are not limited to a specific sequence.

In some examples, the present disclosure provides a composition comprising recombinant αS2-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 3 or a fragment thereof. In some examples, the present disclosure provides a composition comprising recombinant αS2-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 4 or a fragment thereof. It will be understood, however, that any αS2-casein may be suitable for inclusion in a composition of the present disclosure, and that the caseins described herein are not limited to a specific sequence.

In some examples, the present disclosure provides a composition comprising recombinant β-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 5 or a fragment thereof. In some examples, the present disclosure provides a composition comprising recombinant β-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 6 or a fragment thereof. It will be understood, however, that any β-casein may be suitable for inclusion in a composition of the present disclosure, and that the caseins described herein are not limited to a specific sequence.

In some examples, the present disclosure provides a composition comprising recombinant β-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 13 or a fragment thereof. In some examples, the present disclosure provides a composition comprising recombinant β-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 14 or a fragment thereof. It will be understood, however, that any β-casein may be suitable for inclusion in a composition of the present disclosure, and that the caseins described herein are not limited to a specific sequence.

In some examples, the present disclosure provides a composition comprising recombinant κ-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 10 or a fragment thereof. In some examples, the present disclosure provides a composition comprising recombinant κ-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 11 or a fragment thereof. It will be understood, however, that any κ-casein may be suitable for inclusion in a composition of the present disclosure, and that the caseins described herein are not limited to a specific sequence.

In some examples, the present disclosure provides a composition comprising recombinant γ-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 16 or a fragment thereof. In some examples, the present disclosure provides a composition comprising recombinant γ-casein or a fragment thereof having at least 70% sequence identity, at least 75% sequence identity, at least 80% sequence identity, at least 85% sequence identity, at least 90% sequence identity, at least 95% sequence identity, at least 99% sequence identity or 100% sequence identity to the sequence set forth in SEQ ID NO. 17 or a fragment thereof. It will be understood, however, that any κ-casein may be suitable for inclusion in a composition of the present disclosure, and that the caseins described herein are not limited to a specific sequence.

The composition may also comprise other proteins such as whey proteins. The other proteins may be isolated from natural sources or they may be recombinantly produced. The proteins may be recombinantly engineered to comprise specific amino acids, for example, amino acids that provide health benefits or are limiting in an ordinary diet. Suitable whey proteins may include any one or more of β-lactoglobulin, a-lactalbumin, serum albumin, immunoglobulins, lactoferrin, glycomacropeptide and transferrin. In other examples, the composition is essentially free of whey proteins. In some examples, the composition is essentially free of recombinant whey proteins.

The recombinant proteins may be isolated from a microorganism such as a bacterium, a yeast or a fungus. A suitable host for recombinant protein expression may be Trichoderma spp. Other suitable hosts may include a microorganism from the genus Aspergillus, Candida, Fusarium, Hansenula, Kluyveromyces, Pichia, Saccharomyces, Tetrahymena, Trichoderma, Yarrowia or Zygosaccharomyces. A suitable bacterium for recombinant protein expression may be a gram positive bacterium such as Lactococcus lactis or Bacillus subtilis or a gram negative bacterium such as Escherichia coli. Other bacterial hosts may include Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum, Lactobacillus casei, Lactobacillus acidophilus, Lactobacillus plantarum and Synechocystis sp. Those skilled in the art will understand that there are many ways to optimise protein expression and/or purification, for example, by fusing the protein to a tag to assist with purification (eg, strep, His, GST, MBP, SUMO) or secretion (eg, Usp45), by using protease-deficient host cells and by operably linking the coding sequence of the protein to an inducible promoter (eg, nisin, lacZ, T7). The recombinant microbial host cell is preferably generally recognised as safe (GRAS).

In some examples, the recombinant proteins are produced by fermentation of a recombinant microorganism. In some examples, the recombinant proteins are produced using an autocatalytic expression system. In some examples, expression of the recombinant proteins is induced by a chemical such as isopropylthio-β-galactoside (IPTG). For example, a recombinant microorganism may be grown to a suitable optical density (OD), such as OD₆₀₀ 3, 4, 5, 6, 7, 8, 9, 10, 11, 12, 14 or 15, and then treated with IPTG to induce recombinant protein expression. The recombinant microorganism may be cultured (eg, by fermentation) at a temperature that is suitable for growth and protein expression. Fermentation temperature may be between about 25° C. and 40° C., such as between about 30° C. and 40° C., or between about 35° C. and 40° C. or about 37° C. In some examples, the temperature is lowered (eg, to less than 37° C., such as about 15° C., or about 20° C. or about 25° C. or about 30° C. or about 35° C.) after induction of recombinant protein expression. In other examples, the temperature is maintained after induction of protein expression. In some examples, IPTG is added to the culture at a concentration of less than about 2 mM, such as about 1.5 mM, or about 1 mM, or about 0.5 mM, or about 0.4 mM, or about 0.3 mM, or about 0.2 mM, or about 0.1 mM, or about 0.05 mM. A suitable fermentation volume may be between about 100 mL and 10,000,000 L, such as between about 500 mL and 5,000,000 L, or between about 1 L and 5,000,000 L, or between about 5 L and 4,000,000 L, or between about 10 L and 2,500,000 L, or between about 20 L and 1,000,000 L, or between about 50 L and 500,000 L, or between about 100 L and 250,000 L, or between about 100 L and 100,000 L, or between about 100 L and 50,000 L, or between about 200 L and 25,000 L, or between about 500 L and 10,000 L.

Methods for purifying a recombinant protein to obtain a preparation comprising the recombinant protein are well-known in the art (see, for example, Protein Purification, J C Janson and L Ryden, Eds., VCH Publishers, New York, 1989; Protein Purification Methods: A Practical Approach, E L V Harris and S Angel, Eds., IRL Press, Oxford, England, 1989, respectively). A recombinant protein of the present disclosure may be purified on the basis of its molecular weight, for example, by size exclusion/exchange chromatography, ultrafiltration through membranes, gel permeation chromatography (e.g., preparative disc-gel electrophoresis), or density centrifugation. The recombinant protein may be purified on the basis of its surface charge or hydrophobicity/hydrophilicity, for example, by isoelectric precipitation, anion/cation exchange chromatography, isoelectric focusing (IEF), or reverse phase chromatography. The recombinant protein may be purified on the basis of its solubility, for example, by ammonium sulfate precipitation, isoelectric precipitation, surfactants, detergents, or solvent extraction. The recombinant protein may be purified on the basis of its affinity to another molecule, for example, by affinity chromatography, reactive dyes, or hydroxy apatite. Affinity chromatography can include the use of an antibody having a specific binding affinity for the recombinant protein, or a lectin to bind to a sugar moiety on the recombinant milk protein, or any other molecule that specifically binds to the recombinant protein. The recombinant protein can comprise a tag peptide or polypeptide operably fused to its C- or N-terminus to facilitate affinity-based purification of the recombinant protein. For example, the recombinant protein may be fused to a tag (eg, polyhistine tag or a GST tag or a GFP tag) optionally via a cleavable linker. The tag can facilitate affinity purification of the recombinant protein. In some examples, the recombinant protein is secreted by the recombinant microorganism and purified from the culture medium.

Recombinant casein proteins may be isolated from the microorganism by a process comprising: i) lysing the microorganism to produce a cell lysate; ii) heating the cell lysate to produce a heat-treated lysate; iii) centrifuging the heat-treated lysate to obtain a supernatant; iv) adding an acid to the supernatant so as to lower its pH and promote precipitation of the casein protein; and centrifuging the supernatant including precipitated casein proteins to form a pellet of casein protein.

### Lipids

Lipids used in producing dairy-like compositions of the present disclosure are preferably derived from a microorganism. The lipids may be enzymatically modified prior to use in a dairy-like composition. Whole and/or lysed cells of the microorganism may be present in the composition, or the composition may comprise lipids extracted from the microorganism. In some examples, the composition comprises a combination of lipids extracted from the microorganism and whole or lysed cells of the microorganism. The cells, whether intact or lysed, may be prepared by freeze drying or spray drying. The microorganism from which lipids are obtained is, in some examples, not a recombinant microorganism.

The lipids are preferably derived from a yeast, such as Debaryomyces hansenii, Kluyveromyces lactis or Yarrowia lipolytica. Lipids may also be obtained from other microorganisms including bacteria, fungi or algae. The microorganism may be genetically modified, for example, to optimise lipid production, to alter lipid profiles and/or to recombinantly express a protein. The yeast may, for example, express one or more enzymes that enable it to produce a certain type or class of fatty acid. The microorganism is preferably GRAS.

The yeast may be cultured in a batch culture, a fed-batch culture or a continuous culture. The yeast may be grown on media having a carbon: nitrogen (C:N) ratio of between about 10 and about 180, such as between about 10 and 140, or between about 10 and 100, or between about 10 and 80, or between about 10 and 60, or preferably, between about 20 and 60.

Suitable carbon sources may include monosaccharides, disaccharides, polysaccharides, acetate, ethanol, methanol, glycerol, methane, and combinations thereof. Non-limiting examples of monosaccharides include dextrose (glucose), fructose, galactose, xylose, arabinose, and combinations thereof. Non-limiting examples of disaccharides include sucrose, lactose, maltose, trehalose, cellobiose, and combinations thereof. Non-limiting examples of polysaccharides include starch, glycogen, cellulose, amylose, hemicellulose, maltodextrin, and combinations thereof.

Non-limiting examples of assimilable nitrogen sources include anhydrous ammonia, ammonium sulfate, ammonium hydroxide, ammonium nitrate, diammonium phosphate, monoammonium phosphate, ammonium pyrophosphate, ammonium chloride, sodium nitrate, urea, peptone, protein hydrolysates, com steep liquor, com steep solids, spent grain, spent grain extract, and yeast extract. Use of ammonia gas is convenient for large scale operations, and can be employed by bubbling through the aqueous ferment (fermentation medium) in suitable amounts. At the same time, such ammonia can also be employed to assist in pH control.

The lipids obtained from the yeast may comprise saturated fatty acids and unsaturated fatty acids. The ratio of saturated:unsaturated fatty acids may be about 65:35, or about 62:38, or about 60:40: or about 55:45, or about 52:48, or about 51:49, or about 50:50, or about 30:70, or about 20:80, or about 10:90. The saturated fatty acids may include myristic acid (14:0), palmitic acid (16:0) and/or stearic acid (18:0). The unsaturated fatty acids may include oleic acid (18:1), linoleic acid (18:2) and/linolenic acid (18:3).

Lipids may be present in the composition at a concentration of between about 1% and about 50%, such as between about 1% and 40%, or between about 1% and 35%, or between about 1% and 30%, or between about 1% and 29%, or between about 1% and 28%, or between about 1% and 27%, or between about 1% and 26%, or between about 1% and 25%, or between about 1% and 24%, or between about 1% and 23%, or between about 1% and 22%, or between about 1% and 21%, or between about 1% and 20%, or between about 2% and 20%, or between about 2.5% and 20%, or between about 3% and 20%, or between about 3% and 19%, or between about 3% and 18%, or between about 3% and 17%, or between about 3% and 16%, or between about 3% and 15%, or between about 3% and 14%, or between about 3% and 13% or between about 3% and 12%, or between about 3% and 11%, or between about 3% and 10%, or about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5% or about 20%.

Lipids are preferably derived from a microorganism, such as a yeast, but may also be derived from other sources such as plant oil. In some examples, the composition may comprise between about 1% and about 25% microbial lipids, such as between about 1% and 20%, or between about 1% and 15%, or between about 1% and 10%, or between about 2% and 10%, or between about 3% and 10%, or between about 4% and 10%, or between about 5% and 10% microbial lipids. The composition may further comprise between about 1% and 45% plant-derived lipids, such as between about 1% and 40%, or between about 1% and 35%, or between about 1% and 30%, or between about 1% and 25%, or between about 5% and 25%, or between about 5% and 20%, or between about 5% and 15%, or between about 5% and 10% plant-derived lipids. Microbial lipids may provide the composition with certain volatile compounds, metabolites, antioxidants, carotenoids, aromas and/or flavours, and so their concentration in the composition may be selected so as to achieve a desirable taste or smell. Microbial lipids may also contribute to texture, such as in the formation of a curd-like matrix. In some examples, the dairy-like composition of the present disclosure is in the form of a curd.

In some examples, the microbial lipids comprise about 5% to 35% palmitic acid (16:0), such as between about 7.5% and 35%, or between about 9% and 35%, or between about 9% and 32.5%, or between about 9% and 30%, or between about 10% and 30%, or between about 15% and 30%, or between about 17.5% and 30%, or between about 20% and 30% palmitic acid.

In some examples, the microbial lipids comprise about 10% to 45% oleic acid (18:1), such as between about 10% and 35%, or between about 15% and 32.5%, or between about 20% and 32.5% oleic acid.

In some examples, the microbial lipids comprise about 5% to 20% stearic acid (18:0), such as between about 7.5% and 20%, or between about 7.5% and 15%, or between about 10% and 15%, or between about 11% and 15% stearic acid.

In some examples, the microbial lipids comprise about 5% to 35% linoleic acid (18:2), such as about 5% to 30%, or between about 5% and 25%, or between about 5% and 20%, or between about 5% and 15%, or between about 5% and 10% or between about 7.5% and 10% linoleic acid.

In some examples, the microbial lipids comprise less than 10% linolenic acid (18:3), such as less than about 9%, less than about 8%, less than about 7%, less than about 6%, less than about 5%, less than about 4%, less than about 3%, less than about 2% or less than about 1% linolenic acid.

In some examples, the microbial lipids comprise less than 15% myristic acid (14:0), such as less than about 9%, less than about 8%, less than about 7%, less than about 6%, less than about 5%, less than about 4%, less than about 3%, less than about 2%, less than about 1% or less than about 0.5% myristic acid. In some examples, the microbial lipids comprise about 1% to about 15% myristic acid, such as between about 3% and 15% myristic acid, or between about 5% and 15% myristic acid, or between about 7.5% and 15% myristic acid, or between about 10% and 15% myristic acid.

### Additional Components and Uses

The dairy-like compositions described herein may comprise one or more other components such as a plant oil, non-dairy protein, plant protein, carbohydrate, emulsifier, salt, acid, base, pH buffer, sweetening agent, flavour, preservative, vitamin, mineral, antioxidant, texturing/mouthfeel agent, colouring agent, starch, gum, aroma agent, sodium aluminium phosphate, gelling agent or thickening agent.

Suitable plant oils may include sunflower oil, coconut oil, mustard oil, peanut oil, canola oil, com oil, cottonseed oil, flax seed oil, olive oil, palm oil, rapeseed oil, safflower oil, sesame oil, soybean oil, almond oil, beech nut oil, brazil nut oil, cashew oil, hazelnut oil, macadamia nut oil, mongongo nut oil, pecan oil, pine nut oil, pistachio nut oil, walnut oil, avocado oil or grape oil.

The compositions described herein may be used as a dairy substitute in food products such as cheese, cream, sour cream, butter, margarine, spreads, ice cream, skyr, leben, kefir, lassi, milk, a milk-based drink, coffee whitener, yoghurt, custard, infant formula, curd, a dairy powder or a dietary supplement. The compositions may be used as a “dairy concentrate” that can be mixed with an aqueous solution to produce a dairy-like product ready for consumption. The milk-free compositions, or the components described herein, may also be used as a flavouring agent or a seasoning. In some examples, the composition is essentially free of lactose. In some examples, the dairy-like composition forms a curd-like matrix.

In some examples, the present disclosure provides a dairy-like composition comprising:
between about 1% and 40% by weight of one or more recombinant casein proteins or fragments thereof; and
between about 1% and 45% by weight microbial lipids. It will be understood that the compositions described herein preferably comprise an aqueous solution such as water.

In some examples, the present disclosure provides a dairy-like composition comprising:
between about 1% and 40% by weight of one or more recombinant casein proteins or fragments thereof;
between about 1% and 10% by weight microbial lipids; and
between about 3% and 40% by weight plant lipids.

In some examples, the present disclosure provides a dairy-like composition comprising:
between about 1% and 40% by weight of one or more recombinant casein proteins or fragments thereof; and
between about 1% and 45% by weight microbial lipids, wherein the microbial lipids comprise myristic acid, palmitic acid, stearic acid, oleic acid and linoleic acid.

In some examples, the present disclosure provides a dairy-like composition comprising:
between about 1% and 40% by weight of one or more recombinant casein proteins or fragments thereof; and
between about 1% and 45% by weight microbial lipids, wherein the microbial lipids comprise palmitic acid, stearic acid, oleic acid and linoleic acid.

In some examples, the present disclosure provides a dairy-like composition comprising:
between about 1% and 40% by weight of one or more recombinant casein proteins or fragments thereof;
between about 0.5% and 5% by weight microbial lipids; and
between about 1% and 10% by weight total lipids. It will be understood that the total lipids includes microbial lipids and lipids obtained from other sources such as plant oils.

In some examples, the present disclosure provides a dairy-like composition comprising:
between about 1% and 40% by weight of one or more recombinant casein proteins or fragments thereof;
between about 1% and 10% by weight microbial lipids; and
between about 5% and 70% by weight total lipids.

In some examples, the present disclosure provides a dairy-like composition comprising:
between about 10% and 20% by weight of one or more recombinant casein proteins or fragments thereof;
between about 1% and 10% by weight microbial lipids; and
between about 5% and 50% by weight total lipids.

In some examples, the present disclosure provides a dairy-like composition comprising:
between about 10% and 20% by weight of one or more recombinant casein proteins or fragments thereof;
between about 2.5% and 10% by weight microbial lipids; and
between about 5% and 40% by weight total lipids.

In some examples, the present disclosure provides a dairy-like composition comprising:
between about 10% and 20% by weight of one or more recombinant casein proteins or fragments thereof;
between about 1% and 10% by weight microbial lipids; and
between about 5% and 50% by weight total lipids,
wherein the lipids comprise between about 5% and 30% by weight palmitic acid, and/or between about 2% and 20% by weight stearic acid, and/or between about 5% and 45% by weight oleic acid, and/or between about 5% and 35% by weight linoleic acid.

In some examples, the present disclosure provides a dairy-like composition comprising:
between about 10% and 20% by weight of one or more recombinant casein proteins or fragments thereof;
between about 1% and 10% by weight microbial lipids; and
between about 5% and 50% by weight total lipids,
wherein the microbial lipids comprise between about 20% and 30% by weight palmitic acid, and/or between about 5% and 15% by weight stearic acid, and/or between about 25% and 35% by weight oleic acid, and/or between about 5% and 15% by weight linoleic acid.

In some examples, the present disclosure provides a dairy-like composition comprising:
between about 1% and 30% by weight of one or more recombinant casein proteins or fragments thereof; and
between about 1% and 20% by weight microbial lipids,
wherein the one or more casein proteins and the microbial lipids are produced by different microorganisms.

In some examples, the present disclosure provides a dairy-like composition comprising:
between about 1% and 40% by weight of one or more recombinant casein proteins or fragments thereof; and
between about 1% and 40% by weight yeast lipids, wherein at least some of the lipids are extracted from yeast cells.

In some examples, the present disclosure provides a dairy-like composition comprising:
between about 1% and 40% by weight of one or more recombinant casein proteins or fragments thereof; and
between about 1% and 45% by weight yeast lipids,
wherein the composition comprises whole yeast cells comprising the yeast lipids.

In some examples, the present disclosure provides a dairy-like composition comprising:
between about 1% and 40% by weight of one or more recombinant casein proteins or fragments thereof, wherein the recombinant casein proteins are isolated from E. coli or a species of Trichoderma; and
between about 1% and 45% by weight yeast lipids.

In some examples, the present disclosure provides a dairy-like composition comprising:
between about 1% and 40% by weight of one or more recombinant casein proteins or fragments thereof, wherein the recombinant casein proteins are isolated from E. coli or a species of Trichoderma; and
between about 1% and 45% by weight yeast lipids, wherein the yeast is not recombinant.

In some examples, the present disclosure provides a dairy-like composition comprising:
between about 1% and 40% by weight of one or more recombinant casein proteins or fragments thereof, wherein the recombinant casein proteins are isolated from E. coli or a species of Trichoderma; and
between about 1% and 35% by weight yeast lipids, wherein the yeast is a species of Debaryomyces, Kluyveromyces or Yarrowia.

In some examples, the present disclosure provides a dairy-like composition comprising:
between about 1% and 30% by weight of one or more recombinant casein proteins or fragments thereof, wherein the recombinant casein proteins are isolated from E. coli or a species of Trichoderma; and
between about 1% and 30% by weight yeast lipids, wherein the yeast is a species of Debaryomyces.

In some examples, the present disclosure provides a dairy-like composition comprising:
between about 15% and 25% by weight of one or more recombinant casein proteins or fragments thereof;
between about 20% and 30% by weight lipids, wherein the lipids comprise yeast lipids and optionally plant lipids such as coconut oil and/or sunflower oil; and
between 2% and 10% by weight starch. The composition may further comprise sodium aluminium phosphate basic. The compositions described herein may be formed in an aqueuous solution such as water.

### Methods of Production

The present disclosure also provides methods of producing dairy-like compositions comprising one or more recombinant casein proteins and microbial lipids. The casein proteins and lipids are typically mixed in an aqueous solution to form a milk-free, dairy-like composition.

In some examples, the mixing is performed at a speed that will not negatively affect the properties of the composition, such as a speed of about 60 RPM, about 100 RPM, about 200 RPM, about 300 RPM, about 400 RPM, about 500 RPM, about 600 RPM, about 700 RPM, about 800 RPM, about 900 RPM, about 1000 RPM, or more. The mixing may be performed at a temperature of between about 50° C. and 90° C., such as between about 50° C. and 85° C., or between about 55° C. and 85° C., or between about 55° C. and 80° C., or between about 60° C. and 80° C., or between about 65° C. and 80° C., such as about 70° C. or about 75° C. The composition may be mixed at this temperature for a period of between about 1 minute and 30 minutes, such as between about 2 minutes and 25 minutes, or between about 3 minutes and 25 minutes, or between about 4 minutes and 25 minutes, or between about 5 minutes and 25 minutes, or between about 5 minutes and 20 minutes, or between about 10 minutes and 20 minutes, or about 15 minutes. The composition may be formed by mixing the ingredients while the temperature is ramped to, for example, about 50° C., about 55° C., about 60° C., about 65° C., about 70° C., about 75° C. or about 80° C., and then further mixed at that temperature for at least about 5 minutes, about 6 minutes, about 7 minutes, about 8 minutes, about 9 minutes or about 10 minutes or longer.

The composition may be poured into a mould, such as a cube-shaped, cylindrical-shaped, triangular prism-shaped, spherical-shaped, cone-shaped, or rectangular prism-shaped mould. The composition may then be covered and stored, for example at a temperature below 20° C., such as about 15° C., or about 10° C. or about 7.5° C. or about 5° C. or about 4° C. or about 3° C. or about 2° C. or about 1° C. or about 0° C. or below 0° C.

Recombinant casein proteins may be isolated from the microorganism by a process comprising:
i) lysing the microorganism to produce a cell lysate;
ii) heating the cell lysate at a temperature of between about 60° C. and 100° C. to produce a heat-treated lysate;
iii) centrifuging the heat-treated lysate to obtain a supernatant;
iv) adding an acid to the supernatant so as to lower its pH and promote precipitation of the casein protein; and
v) centrifuging the supernatant including precipitated casein proteins to form a pellet of casein protein.

Preferably, the acid is added to the supernatant such that the pH of the supernatant is approximately equal to the isoelectric point of the casein protein.

The pellet of casein protein may optionally be resuspended or washed with water. The mixture of casein and water may then be centrifuged to form a washed pellet of casein protein. In some examples, DNase is not added to the purified casein.

The wet casein proteins may be freeze dried or spray dried prior to use in a dairy-like composition.

In some examples, the present disclosure provides a recombinant casein powder obtained by a process comprising:
i) culturing a microorganism recombinantly expressing casein;
ii) lysing the microorganism to produce a cell lysate;
iii) heating the cell lysate at a temperature of between about 60° C. and 100° C. to produce a heat-treated lysate;
iv) centrifuging of heat-treated lysate to obtain a supernatant;
v) adding an acid to the supernatant so as to lower its pH and promote precipitation of the casein;
vi) centrifuging the supernatant including precipitated casein to form a pellet of casein;
vii) resuspending the pellet of casein in an aqueous solution;
viii) centrifuging the aqueous solution to form a washed pellet of casein;
ix) resuspending the washed pellet of casein in an aqueous solution; and
x) optionally freeze drying or spray drying the casein to produce the recombinant casein powder

### Items of the Present Disclosure

Set forth below are non-limiting differentiation media, methods, cells and food compositions of the present disclosure.
1. A dairy-like composition comprising:
   one or more recombinant casein proteins or fragments thereof; and
   microbial lipids.
2. The composition of Item 1 wherein the composition is milk-free.
3. The composition of Item 1 or Item 2 wherein the one or more recombinant casein proteins are selected from the group consisting of αS1-casein, αS2-casein, β-casein and κ-casein.
4 The composition of any one of Items 1 to 3 wherein the one or more casein proteins comprise αS1-casein and/or β-casein.
5. The composition of any one of Items 1 to 4 wherein at least one of the recombinant casein proteins is expressed in a microorganism from a codon optimised gene.
6. The composition of any one of Items 1 to 5 wherein the one or more casein proteins are produced by a microorganism.
7. The composition of any one of Items 1 to 6 wherein the one or more casein proteins and the microbial lipids are produced by different microorganisms.
8. The composition of any one of Items 1 to 7 wherein the one or more casein proteins and the microbial lipids are produced by different species of microorganisms
9. The composition of any one of Items 1 to 8 wherein the recombinant casein proteins are isolated from E. coli or a species of Trichoderma.
10. The composition of any one of Items 1 to 9 wherein at least one of the recombinant casein proteins is isolated from a microorganism by a process comprising:
   i) lysing the microorganism to produce a cell lysate;
   ii) heating the cell lysate to produce a heat-treated lysate;
   iii) centrifuging the heat-treated lysate and obtaining a supernatant;
   iv) adding an acid to the supernatant so as to lower its pH and promote precipitation of the casein protein; and
   v) centrifuging the supernatant to form a pellet of casein protein.
11. The composition of Item 10 wherein the process further comprises:
   vi) resuspending the pellet of casein protein in an aqueous solution; and
   vii) freeze drying or spray drying the resuspended casein protein.
12. The composition of any one of Items 1 to 11 wherein the microbial lipids are produced by a microorganism that is not recombinant.
13. The composition of any one of Items 1 to 12 wherein the microbial lipids are yeast lipids.
14. The composition of Item 13 wherein the yeast is Debaryomyces hansenii, Kluyveromyces lactis or Yarrowia lipolytica.
15. The composition of Item 13 wherein the yeast is Debaryomyces hansenii.
16. The composition of any one of Items 13 to 15 wherein the composition comprises whole yeast cells, and wherein the yeast cells comprise the lipids.
17. The composition of Item 16 wherein the yeast cells are obtained by spray drying or freeze drying.
18. The composition of any one of Items 13 to 17 wherein the composition comprises lysed spray dried yeast cells, and wherein the yeast cells comprise the lipids.
19. The composition of any one of Items 13 to 18 wherein the composition comprises lipids extracted from yeast cells.
20. The composition of any one of Items 1 to 19 wherein the composition comprises recombinant casein proteins at a concentration of between 5% and 25%.
21. The composition of any one of Items 1 to 20 wherein the composition comprises microbial lipids at a concentration of between 1% and 20%.
22. The composition of any one of Items 1 to 21 wherein the composition comprises microbial lipids at a concentration of between 1% and 5%.
23. The composition of any one of Items 1 to 22 wherein the microbial lipids comprise palmitic acid, stearic acid, oleic acid, linoleic acid and linolenic acid.
24. The composition of any one of Items 1 to 23 wherein the composition further comprises a plant oil or a plant protein.
25. The composition of any one of Items 1 to 24 wherein the composition comprises between about 4% and 40% lipids, and wherein a fraction of the lipids are microbial lipids.
26. The composition of Item 25 comprising between about 1% and 5% microbial lipids and between about 5% and 40% total lipids.
27. A method of producing a dairy-like composition the method comprising mixing one or more recombinant casein proteins or fragments thereof with microbial lipids.
28. The method of Item 27 wherein the composition is milk-free.
29. The method of Item 27 or Item 28 wherein the mixing is performed at a temperature of between about 70° C. and 80° C.
30. The method of any one of Items 27 to 29 wherein the microbial lipids are yeast lipids.
31. The method of Item 30 wherein the yeast is Debaryomyces hansenii, Kluyveromyces lactis or Yarrowia lipolytica.
32. The method of Item 30 or Item 31 wherein the lipids are added to the composition in the form of whole yeast cells comprising the lipids.
33. The method of any one of Items 30 to 32 wherein the lipids are added to the composition in the form of lysed yeast cells comprising the lipids.
34. The method of Item 32 or Item 33 wherein the yeast cells are prepared by spray drying or freeze drying.
35. The method of any one of Items 30 to 34 wherein the lipids are extracted from yeast cells.
36. The method of any one of claims 30 to 35 wherein the one or more recombinant casen proteins are isolated from bacteria such as E. coli or a species of Trichoderma.
37. A food or beverage product comprising a dairy-like composition of any one of Items 1 to 26.

### EXAMPLES

### Recombinant Protein Production

Nucleic acid sequences encoding the Alpha S1 (AS1), Beta Casein (BCn), Alpha S2 (AS2) and Kappa (KP) casein proteins were synthesized with their codon usage preferences suited for expression in the bacterium Escherichia coli (E. coli). The AS1 protein sequence contains 214 amino acid residues. It has a theoretical molecular weight (Mwt) of 24528.94 Da and an isoelectric point (pI) of 4.6974. The AS1 gene sequence is 645 bp in length. The BCn protein sequence contains 224 amino acid residues. It has a Mwt of 25107.33 Da and a pl of 5.080. The Beta casein gene is 675 bp in length. The AS2 protein sequence has 222 amino acid residues. The AS2 gene sequence is 669 bp long. The Kappa protein sequence has 190 amino acids, a Mwt of 21269.35 Da and a pI of 6.7. The KP gene sequence is 573 bp in length.

The following casein sequences were used:

Alpha S1
(SEQ ID NO. 1)
MKLLILTCLVAVALARPKHPIKHQGLPQEVLNENLLRFFVAPFPEVFGKEKVNELSKDIG
SESTEDQAMEDIKQMEAESISSSEEIVPNSVEQKHIQKEDVPSERYLGYLEQLLRLKKYK
VPQLEIVPNSAEERLHSMKEGIHAQQKEPMIGVNQELAYFYPELFRQFYQLDAYPSGAWY
YVPLGTQYTDAPSFSDIPNPIGSENSEKTTMPLW
Mature Alpha S1 protein is bold.
(SEQ ID NO. 2)
Alpha S2
(SEQ ID NO. 3)
MKFFIFTCLLAVALAKNTMEHVSSSEESIISQETYKQEKNMAINPSKENLCSTFCKEVVR
NANEEEYSIGSSSEESAEVATEEVKITVDDKHYQKALNEINQFYQKFPQYLQYLYQGPIV
LNPWDQVKRNAVPITPTLNREQLSTSEENSKKTVDMESTEVFTKKTKLTEEEKNRLNFLK
KISQRYQKFALPQYLKTVYQHQKAMKPWIQPKTKVIPYVRYL
Mature Alpha S2 protein is bold.
(SEQ ID NO. 4)
Beta casein
(SEQ ID NO. 5)
MKVLILACLVALALARELEELNVPGEIVESLSSSEESITRINKKIEKFQSEEQQQTEDEL
QDKIHPFAQTQSLVYPFPGPIPNSLPQNIPPLTQTPVVVPPFLQPEVMGVSKVKEAMAPK
HKEMPFPKYPVEPFTESQSLTLTDVENLHLPLPLLQSWMHQPHQPLPPTVMFPPQSVLSL
SQSKVLPVPQKAVPYPQRDMPIQAFLLYQEPVLGPVRGPFPIIV
Mature Beta casein protein is bold.
(SEQ ID NO. 6)
Alpha S1 synthetic gene sequence.
(SEQ ID NO. 7)
ATGAAGCTGCTGATCCTGACCTGCCTGGTGGCCGTGGCCCTGGCCCGTCCGAAGCATCCGATTAAGCACCAGGGTC
TGCCGCAAGAGGTTCTGAACGAAAACCTGCTGCGTTTCTTTGTGGCGCCGTTCCCGGAAGTGTTCGGCAAGGAGAA
AGTGAACGAACTGAGCAAGGACATCGGCAGCGAGAGCACCGAGGACCAGGCGATGGAGGATATTAAACAAATGGAG
GCGGAGAGCATCAGCAGCAGCGAGGAAATTGTGCCGAACAGCGTTGAACAGAAGCACATCCAAAAAGAGGATGTGC
CGAGCGAACGTTACCTGGGCTATCTGGAGCAGCTGCTGCGTCTGAAGAAATACAAGGTTCCGCAACTGGAAATCGT
TCCGAACAGCGCGGAGGAACGTCTGCACAGCATGAAAGAGGGTATCCACGCGCAGCAAAAAGAACCGATGATTGGC
GTTAACCAGGAGCTGGCGTACTTCTATCCGGAACTGTTCCGTCAGTTTTACCAACTGGACGCGTATCCGAGCGGTG
CGTGGTACTATGTGCCGCTGGGCACCCAATATACCGATGCGCCGAGCTTTAGCGACATTCCGAACCCGATTGGCAG
CGAAAATAGCGAAAAAACCACCATGCCGCTGTGGTAA
Alpha S2 synthetic gene sequence
(SEQ ID NO. 8)
ATGAAGTTCTTCATCTTCACCTGCCTGCTGGCCGTGGCCCTGGCCAAGAACACCATGGAGCACGTGAGCAGCAGCG
AGGAGAGCATCATCAGCCAGGAGACCTACAAGCAGGAGAAGAACATGGCCATCAACCCCAGCAAGGAGAACCTGTG
CAGCACCTTCTGCAAGGAGGTGGTGAGGAACGCCAACGAGGAGGAGTACAGCATCGGCAGCAGCAGCGAGGAGAGC
GCCGAGGTGGCCACCGAGGAGGTGAAGATCACCGTGGACGACAAGCACTACCAGAAGGCCCTGAACGAGATCAACC
AGTTCTACCAGAAGTTCCCCCAGTACCTGCAGTACCTGTACCAGGGCCCCATCGTGCTGAACCCCTGGGACCAGGT
GAAGAGGAACGCCGTGCCCATCACCCCCACCCTGAACAGGGAGCAGCTGAGCACCAGCGAGGAGAACAGCAAGAAG
ACCGTGGACATGGAGAGCACCGAGGTGTTCACCAAGAAGACCAAGCTGACCGAGGAGGAGAAGAACAGGCTGAACT
TCCTGAAGAAGATCAGCCAGAGGTACCAGAAGTTCGCCCTGCCCCAGTACCTGAAGACCGTGTACCAGCACCAGAA
GGCCATGAAGCCCTGGATCCAGCCCAAGACCAAGGTGATCCCCTACGTGAGGTACCTGTAG
Beta casein synthetic gene sequence
(SEQ ID NO. 9)
ATGAAGGTGCTGATCCTGGCCTGCCTGGTGGCCCTGGCCCTGGCCCGTGAGCTGGAAGAACTGAACGTGCCGGGTG
AAATCGTTGAGAGCCTGAGCAGCAGCGAGGAAAGCATCACCCGTATCAACAAGAAAATTGAGAAGTTCCAAAGCGA
GGAACAGCAACAGACCGAGGACGAGCTGCAAGATAAAATCCACCCGTTCGCGCAAACCCAGAGCCTGGTGTACCCG
TTTCCGGGTCCGATCCCGAACAGCCTGCCGCAAAACATTCCGCCGCTGACCCAGACCCCGGTGGTTGTGCCGCCGT
TTCTGCAACCGGAAGTGATGGGCGTGAGCAAGGTTAAAGAGGCGATGGCGCCGAAGCACAAAGAAATGCCGTTCCC
GAAGTATCCGGTGGAGCCGTTTACCGAAAGCCAAAGCCTGACCCTGACCGACGTTGAAAACCTGCACCTGCCGCTG
CCGCTGCTGCAAAGCTGGATGCATCAACCGCACCAACCGCTGCCGCCGACCGTGATGTTCCCGCCGCAAAGCGTTC
TGAGCCTGAGCCAGAGCAAGGTGCTGCCGGTTCCGCAGAAAGCGGTTCCGTACCCGCAACGTGATATGCCGATTCA
GGCGTTTCTGCTGTATCAAGAGCCGGTTCTGGGTCCGGTGCGTGGTCCGTTTCCGATTATTGTGTAA

Suitable Kappa casein sequences include the following:

Kappa casein
(SEQ ID NO. 10)
MMKSFFLVVTILALTLPFLGAQEQNQEQPIRCEKDERFFSDKIAKYIPIQYVLSRYPSYG
LNYYQQKPVALINNQFLPYPYYAKPAAVRSPAQILQWQVLSNTVPAKSCQAQPTTMARHP
HPHLSFMAIPPKKNQDKTEIPTINTIASGEPTSTPTTEAVESTVATLEDSPEVIESPPEI
NTVQVTSTAV
Mature Kappa casein protein is bold
(SEQ ID NO. 11)
Kappa casein synthetic gene sequence
(SEQ ID NO. 12)
ATGATGAAATCATTTTTCCTAGTAGTTACAATCCTGGCACTGACCCTGCCGTTTCTGGGT
GCTCAAGAGCAGAACCAAGAGCAACCGATCCGCTGCGAAAAAGATGAACGCTTCTTCAGC
GATAAGATTGCCAAGTACATCCCGATCCAGTATGTGTTGTCTCGTTATCCGAGCTATGGC
CTCAATTATTACCAGCAGAAGCCGGTTGCTTTGATCAATAACCAATTTTTGCCCTACCCG
TACTACGCAAAACCGGCGGCGGTTCGTAGCCCGGCACAGATTCTTCAATGGCAGGTTCTG
TCCAATACCGTTCCGGCGAAAAGCTGTCAGGCGCAGCCGACCACCATGGCGCGTCACCCG
CATCCGCACCTGTCGTTCATGGCGATTCCGCCTAAAAAGAACCAAGACAAGACCGAGATT
CCGACGATTAACACCATCGCGAGCGGTGAACCGACTTCTACGCCGACCACCGAGGCGGTC
GAAAGCACGGTGGCTACCCTGGAAGACTCCCCAGAGGTAATTGAATCCCCGCCAGAGATC
AACACCGTGCAGGTGACCTCAACTGCCGTCTAA

Further suitable casein sequences may include the following:

Beta casein variant
(SEQ ID NO. 13)
MRELEELNVPGEIVESLSSSEESITRINKKIEKFQSEEQQQTEDELQDKIHPFAQTQSLVYPFPGPIPNSLPQNIP
PLTQTPVVVPPFLQPEVMGVSKVKEAMAPKHKEMPFPKYPVEPFTESQSLTLTDVENLHLPLPLLQSWMHQPHQPL
PPTVMFPPQSVLSLSQSKVLPVPQKAVPYPQRDMPIQAFLLYQEPVLGPVRGPFPIIV
Mature beta casein variant sequence is bold
(SEQ ID NO. 14)
Beta casein synthetic gene sequence variant
(SEQ ID NO. 15)
ATGCGCGAACTGGAAGAACTGAACGTGCCGGGCGAAATTGTGGAAAGCCTGAGCAGCAGCGAAGAAAGC
ATTACCCGCATTAACAAAAAAATTGAAAAATTTCAGAGCGAAGAACAGCAGCAGACCGAAGATGAACTG
CAGGATAAAATTCATCCGTTTGCGCAGACCCAGAGCCTGGTGTATCCGTTTCCGGGCCCGATTCCGAAC
AGCCTGCCGCAGAACATTCCGCCGCTGACCCAGACCCCGGTGGTGGTGCCGCCGTTTCTGCAGCCGGAA
GTGATGGGCGTGAGCAAAGTGAAAGAAGCGATGGCGCCGAAACATAAAGAAATGCCGTTTCCGAAATAT
CCGGTGGAACCGTTTACCGAAAGCCAGAGCCTGACCCTGACCGATGTGGAAAACCTGCATCTGCCGCTG
CCGCTGCTGCAGAGCTGGATGCATCAGCCGCATCAGCCGCTGCCGCCGACCGTGATGTTTCCGCCGCAG
AGCGTGCTGAGCCTGAGCCAGAGCAAAGTGCTGCCGGTGCCGCAGAAAGCGGTGCCGTATCCGCAGCGC
GATATGCCGATTCAGGCGTTTCTGCTGTATCAGGAACCGGTGCTGGGCCCGGTGCGCGGCCCGTTTCCG
ATTATTGTGTAA
Bovine gamma casein
(SEQ ID NO. 16)
MKIEKFQSEEQQQTEDELQDKIHPFAQTQSLVYPFPGPIPNSLPQNIPPLTQTPVVVPPFLQPEVMGVS
KVKEAMAPKHKEMPFPKYPVEPFTESQSLTLTDVENLHLPLPLLQSWMHQPHQPLPPTVMFPPQSVLSL
SQSKVLPVPQKAVPYPQRDMPIQAFLLYQEPVLGPVRGPFPIIV
Mature gamma casein sequence is bold
(SEQ ID NO. 17)
Bovine gamma casein synthetic gene sequence
(SEQ ID NO. 18)
ATGAAGATCGAGAAGTTCCAGAGCGAGGAGCAGCAGCAGACCGAGGACGAGCTGCAGGACAAGATCCACCCCTTCG
CCCAGACCCAGAGCCTGGTGTACCCCTTCCCCGGCCCCATCCCCAACAGCCTGCCCCAGAACATCCCCCCCCTGAC
CCAGACCCCCGTGGTGGTGCCCCCCTTCCTGCAGCCCGAGGTGATGGGCGTGAGCAAGGTGAAGGAGGCCATGGCC
CCCAAGCACAAGGAGATGCCCTTCCCCAAGTACCCCGTGGAGCCCTTCACCGAGAGCCAGAGCCTGACCCTGACCG
ACGTGGAGAACCTGCACCTGCCCCTGCCCCTGCTGCAGAGCTGGATGCACCAGCCCCACCAGCCCCTGCCCCCCAC
CGTGATGTTCCCCCCCCAGAGCGTGCTGAGCCTGAGCCAGAGCAAGGTGCTGCCCGTGCCCCAGAAGGCCGTGCCC
TACCCCCAGAGAGACATGCCCATCCAGGCCTTCCTGCTGTACCAGGAGCCCGTGCTGGGCCCCGTGAGAGGCCCCT
TCCCCATCATCGTG

E. coli strains NiCo21 (DE3), Tuner (DE3)-Novagen, Origami B (DE3)-Novagen and SHuffle T7 were used as protein production hosts. The pETDuet 1, pACYC-Duet 1 and pET-22b (+) plasmids were used for cloning and expression of the caseins.

All strains were propagated on Luria broth (LB)-agar (10 g/L Bacto tryptone, 5 g/L yeast extract, 5 g/L NaCl, 15 g/L bacteriological agar) plates containing either Ampicillin (100 μg/mL) or Chloramphenicol (34 μg/mL), 37° C., 12-13 hrs. For liquid growth medium, either LB broth or Terrific broth (12 g/L Bacto tryptone, 24 g/L yeast extract, 12.54 g/L K₂HPO₄, 2.31 g/L KH₂PO₄, 10 g/L Glycerol), and/or NMM (Soy peptone 20 g/L, Yeast extract, 10 g/L, Glycerol 30 g/L, KH₂PO₄ 0.75 g/L, Na₂HPO₄ 0.75 g/L, MgSO₄.7H₂O, 0.62 g/L, KH₂PO₄, 0.085 g/L, NaCl 10 g/L, NH₄Cl, 0.7 g/L, K₂SO₄, 0.085 g/L, MgSO₄.7H₂O, 0.62 g/L, Fe(III) citrate, 0.0125 g/L, MnCl₂.7H₂O, 0.015 g/L, Zn(CH₃COO)₂.2H₂O, 0.0013 g/L, H₃BO₃, 0.0025 g/L, Na₂MoO₄.2H₂O, 0.0025 g/L, CoCl₂.6H₂O, 0.0025 g/L, CuCl₂.2H₂O, 0.0015 g/L, Na₂EDTA, 0.0014 g/L) was used for growing of each strain containing the appropriate antibiotic, 37° C., 200-250 rpm, 12-13 hrs.

Selected recombinant strain/s were grown in LB medium, 37° C. 12-13 hrs, and used as a seed for protein production the next day. Either Terrific broth (TB) or NMM medium was used for protein production.

Cultivations were performed in 1-10 L fermenters. A seed culture was grown in LB broth to provide a 1-5% (v/v) inoculum into the either TB or NMM medium for protein production. Following inoculation, the cultures were grown at 37° C., 250 rpm, and the optical density (OD₆₀₀ₙₘ) was monitored. Once the OD₆₀₀ value reached 9, Isopropylthio-β-galactoside (IPTG) was added to a final concentration of 0.2 mM. The cultures were continued and stopped after 3 hours.

Cultivations were scaled up to 1 L and 10 L total working volume (TWV) fermenters. The following conditions were employed for batch runs:
Fermentation initial conditions:
   Agitation/TIPSPD (Rushton): 177 cm/sec
   Temperature: 37° C.
   pH: No control in TB. ˜7 for NMM
   Dissolved oxygen set point: 30%
   Airflow: 0.5 VVM
   Fermentation time: 6-8 hrs total
   Fermentation working volume: 7.15 L
   Antifoam: SILFAX D5100 (1:1) or Antifoam 204 (1:1-1:10) with MQ H₂O
Cascade conditions:
   TIPSPD: 177-393 cm/sec
   Airflow: 0.5-1.0 VVM
   DO set at 30%
      Cultivations were also performed at room temperature (about 22-23° C.) with cells being harvested following about 28 hours elapsed fermentation time (EFT). Cultivations using NMM medium were performed for about 11-18 hours EFT.

### Downstream Processing and Protein Isolation

Following 3 hours post induction (or 16 hours when cells were grown at room temperature), fermentation was terminated and the downstream process started in order to isolate the protein/s of interest.

Cells were removed from the fermenter into 1 L containers, and centrifuged at 7500 rpm, 15 mins, 10° C. Cells were fully resuspended to a 3^{rd} of its original volume in TES buffer (50 mM Tris, 5 mM EDTA, 2.922 g/L NaCl, pH 7.5).

Cell suspensions were then lysed at 28 Kpsi using a homogenizer with 2-3 passes or until cells were fully lysed. A modified lysis buffer was used (150 mM NaCl, 1 mM EDTA, 1% Tween 20, 5% glycerol).

Following cell lysis, the cell suspensions were heat treated at 70-80° C. for 1-2 hrs, followed by centrifugation at 7500 rpm, 15 mins, 10° C. Clear supernatant was removed into a new container. 10% acetic acid was added with gentle stirring until the pH of the solution containing casein proteins reached 4.6-5.0, causing the casein proteins to precipitate out of solution. The solution was left at 22-23° C. for 30 mins. The casein solution was centrifuged at 7500 rpm, 15 mins, 10° C., and the casein protein pellet was resuspended/washed with MQ-H₂O. This step was performed 2-3 times. Following the final wash, the MQ-H₂O was removed, and caseins were weighed and expressed as wet caseins.

### Freeze and Spray Drying

The wet caseins were either freeze dried or spray dried using the following conditions:
Freeze drying
   1. Freeze the wet casein pellet at −20° C. for 24 hours.
   2. Place the frozen casein pellet in a freeze dryer (condenser temperature −85° C. with vacuum pump) until the moisture content reduce to 10-12%.
Spray drying (Buchi Mini Spray Dryer B-290)
   Inlet temperature: 130° C.
   Outlet temperature: 60-70° C.
   Pump rate: 30%
   Q flow: 40 mM
   Volume: 300 mL H₂O containing ˜100 g wet weight of protein
   Feeding rate: 8 mL/min

### Lipid Production

Debaryomyces hansenii CF10, Kluyveromyces lactis CF3, and Yarrowia lypolytica CF4 were all tested for lipid production. All yeast strains were propagated on YPD agar plates (10 g/L yeast extract, 20 g/L peptone, 20 g/L dextrose, and 20 g/L bacteriological agar), 28° C., 12-13 hours.

Strains were cultivated in YPD broth (without the agar), 28° C., 200-250 rpm, 12-168 hours. All yeasts were tested for lipid production under different carbon to nitrogen ratios, 28 C, 200-250 rpm, 12-168 hours. Initial tests were carried out using a minimal salts-based medium with glucose as the carbon source.

### Reagents

Stock glucose solution (256 g/L):
   Weigh 76.8 g glucose in distilled water and transfer quantitatively to a 300 mL volumetric flask and make up to 300 mL with distilled water. Label and store at room temperature for 3 months.
Stock basal media:
   Weigh 1.066 g yeast extract, 3.2 g MgSO₄.7H₂O, 14.92 g KH₂PO₄, and 5.32 g Na₂HPO₄ and transfer quantitatively to a 500 mL volumetric flask and make up to 1500 mL with distilled water.
Stock (NH₄)₂SO₄ solution (30 g/L):
   Weigh 3.0 g of (NH₄)₂SO₄ in distilled water and transfer quantitatively to a 250 mL volumetric flask and make up to 100 mL with distilled water. Label and store at room temperature for 3 months.
Basal media for screening activity:
   The stock basal media and (NH₄)₂SO₄ solution is mixed and diluted in distilled water, as follows, to make a total volume of 400 mL in 500 mL volumetric flasks:

| | Volume of | Volume of | Volume of stock | Volume of | Total |
|---|---|---|---|---|---|
| C:N | stock basal | stock glucose | (NH₄)₂SO₄ sol. | distilled water | volume |
| Ratio | media (mL) | sol. (mL) | (mL) | (mL) | (mL) |
| 20 | 300 | 50 | 37.3 | 12.7 | 400 |
| 60 | 300 | 50 | 10.4 | 39.5 | 400 |
| 100 | 300 | 50 | 5.066 | 44.93 | 400 |
| 140 | 300 | 50 | 2.76 | 47.24 | 400 |
| 180 | 300 | 50 | 1.48 | 48.52 | 400 |

All basal media were sterilized at 121° C., 30 mins, and aliquoted into flasks. Glucose was filter sterilized through a 0.2 μ filter and added into each flask. All experiments were carried out in replicate when using shake flasks. Later, glucose was replaced with sucrose as the carbon source.

Nile red staining for the detection of intracellular lipid production: Cells were removed during cultivations and checked for intracellular lipid accumulation using Nile red dye.

### Reagents

Phosphate buffered saline (PBS) 10 mM, pH 7.4
   For 1 L, mix the following ingredients: 8 g NaCl, 200 mg KCl, 1.44 g Na₂HPO₄, and 240 mg KH₂PO₄ and adjust volume to 800 mL using distilled water. Adjust pH to 7.4 using HCl and NaOH. Add distilled water until volume is 1 L. Label and store at room temperature for 6 months.
DMSO: PBS (1:1 v/v) solution
   Mix DMSO and PBS 1:1 on volume basis. Mix well. Label and store at room temperature.
Nile red (50 μg mL⁻¹) in acetone
   Weigh 100 μg of Nile red and dissolve in 2 mL of acetone. Label and store in the dark. Prepare fresh before use.

### Reagents

Phosphate buffered saline (PBS) 10 mM, pH 7.4
   For 1 L, the following ingredients were mixed: 8 g NaCl, 200 mg KCl, 1.44 g Na₂HPO₄, and 240 mg KH₂PO₄ and adjust volume to 800 mL using distilled water. Adjust pH to 7.4 using HCl and NaOH. Distilled water added until volume is 1 L. Label and store at room temperature for 6 months.
DMSO: PBS (1:1 v/v) solution
   Mix DMSO and PBS 1:1 on volume basis. Label and store at room temperature.
Nile red (50 μg mL⁻¹) in acetone
   Weigh 100 μg of Nile red and dissolve in 2 mL of acetone. Label and store in the dark. Prepare fresh before use.

### Methods

1. Measure OD₆₀₀ of each grown culture.

2. Adjust cell density in PBS to OD₆₀₀=1.0.
3. Transfer 250 μl of culture to a 96-well black microplate in triplicate. To each well, 25 μl DMSO/PBS (1:1, v/v) and 25 μL of 50 μg mL⁻¹ Nile red are mixed into the culture before the lipid measurement. Final concentration of 5 μg mL⁻¹ Nile red.
4. Initial absorbance reading is at 600 nm; initial fluorescence excitation at 530/25, emission at 590/35; and kinetic reading for 20 min with 60 s interval. The optic position is set to top 50%. Maximal emission values are determined.
Fluorescence data were corrected for variation in cell density by dividing the fluorescence unit by background OD₆₀₀ values.

Nile red staining for fluorescence microscopy: The following method was performed for preparing and imaging of yeast using fluorescence microscopy (Poli et al., 2013. Rev. Bras. Biocienc. 11 203-208; Rostron et al., 2015. Antonie Van Leeuwenhoek 108 97-106).

### Reagents

Nile red (0.1 mg/mL) staining:
Nile red solution was prepared by dissolving 0.1 mg of Nile red in 1 mL of acetone.
PBS buffer 10 mM (pH 7.4)
For 1 L, mix the following ingredients: 8 g NaCl, 200 mg KCl, 1.44 g Na₂HPO₄, and 240 mg KH₂PO₄ and adjust volume to 800 mL using distilled water. Adjust pH to 7.4 using HCl and NaOH. Add distilled water until volume is 1 L. Label and store at room temperature for 6 months.

### Methods

1. Each sample is centrifuged at 2000 rpm for 5 min. Collect the first supernatant and then wash the cell pellet twice with 0.5-1 mL 10 mM PBS buffer.
2. Preparing slides for fluorescence microscopy
3. Prepare a fresh 4% formaldehyde solution by dissolving paraformaldehyde powder into PBS (pH 7.4) using a stirring hot plate with the heater set to a medium setting until the liquid reaches approximately 60° C. As the paraformaldehyde breaks down to formaldehyde it will dissolve. Once the solution is completely dissolved (approximately 30 min), the solution is quickly chilled on ice back to room temperature prior to use.
4. Fix cells for 10 minutes at room temperature with 4% paraformaldehyde in PBS followed by 2-3 washes with PBS to remove excess formaldehyde and stop the fixing reaction.
5. Add 10 μL of formaldehyde fixed cells on top of poly L lysine containing cover slips. Let the cell suspension dry in a fume hood and follow the Nile red staining protocol.
6. Add 10 μL of formaldehyde (or glutaraldehyde) fixed cell suspension onto the middle of the glass slide. Let it dry under the fume hood. Heat fix and follow the Nile red staining protocol.
7. Add 10 μL of original cell suspension (not formaldehyde or glutaraldehyde fixed) onto the middle of the glass slide. Let it dry under the fume hood. Heat fix and follow the Nile red staining protocol.
8. Add 10 μL of Nile red solution to the smear and incubate at room temperature for 5 min.
9. Remove the excess of Nile red using PBS buffer 10 mM (pH 7.4). The washed slide is covered with a coverslip and blotted dry with paper tissue.
10. Wrap each slide with an aluminium foil and place in dark container.
11. Examine at 100× magnification under a fluorescent microscope. A dual excitation interference of excitation filters: FITC (475-490 nm) and TRITC (540-565 nm) is used.
12. Lipids are observed as yellow gold droplets, whose size is visually estimated in relation to cell area.

### Determination of Fatty Acid Profiles by Gas Chromatography-Mass Spectroscopy (GC-MS)

The following reagents and standards were used to determine fatty acid profiles:
Chloroform, high-performance liquid chromatograph (HPLC) grade
Methanol, HPLC grade
Hexane, HPLC grade
Hydrochloric acid (HCl), concentrated (36.5%-38%) (˜12M)
Nonadecanoic acid (C19:0) as the Internal Standard
37-component FAME mix certified reference material (Catalog number CRM47885, Supelco)

Preparation of internal standard:
1. To make up a 10 mg/mL solution, weigh 100 mg of the nonadecanoate (C19:0) and add to a 10 mL, class A, volumetric flask. Bring to volume with HPLC grade hexane and mix.
2. Transfer the hexane-C13:0 mixture into 1.5 mL GC vials and seal immediately with caps. Store the vials at −20° C. for up to 6 months.

Transesterification of samples:
1. Mix a portion of freeze/spray dried yeast biomass (˜50 mg) with 2 mL solvent mixture containing methanol/hydrochloric acid/chloroform (10:1:1, v/v/v) in a 8 mL glass extraction tube with a screw cap.
2. Add 100 μL (1 μg) of the pre-prepared C19:0 internal standard (10 mg/mL) to the extraction vial and vortex well to mix the contents. (This amount may be adjusted to more accurately reflect the estimated fatty acid content of the sample.)
3. Heat the mixture at 90° C. for 1 h after closing the extraction vial to convert microbial oils to fatty acid methyl esters (FAMEs).
4. Remove the vials and cool for at least 15 minutes, but not longer than an hour, at room temperature.
5. Add 1 mL 0.9% NaCl solution to the mixture and mix.

Extraction and preparation of FAMEs
1. Add 0.5 mL hexane to the vial and vortex well to mix the vial contents and let them stand undisturbed at room temperature for at least 1 hour (but not more than 4 hours) to allow the phases to separate.
2. Transfer the hexane phase containing FAMEs (upper phase of the sample) to a 1.5 mL GC vial and dilute if appropriate with hexane.
3. Analyse the samples by injecting 1 μL of sample with a split ratio of 10:1. Using an Agilent 6890 Series Gas Chromatography system equipped with a HP 5973 mass spectrometer detector and a HP-5MS capillary column (Agilent J&W 30 m×0.25 mm×0.25 μm. The injection port temperature: 230° C. Initial column temperature: 90° C. and hold for 1 min, followed by increasing the column temperature at a rate of 15° C./min until 180° C., 5° C./min to 220° C., 10° C./min until 250° C. and hold for 10 min. Use 37 component FAME Mix (Supelco, Sigma-Aldrich) as the standard mix.

Quality control: Internal standard, 37-component FAME Mix as certified reference material, method blanks, calibration blanks, intermediate checks, biological replicates.

### Gravimetric Analysis of Total Lipids

Microbial oil content was determined gravimetrically using the modified Bligh and Dyer method:
1. Weigh 40 mg freeze-dried biomass to a 2.0 mL centrifuge tube.
2. Add stainless steel beads (one big and three small ones) into the tube and lyse cells using TissueLyser (Qiagen) at 30 Hz for 4 min (two minutes each side).
3. Add 0.24 mL water, 0.3 mL chloroform and 0.6 mL CH₃OH and homogenise for 2 min (2×1 min) and centrifuge at 12000 rpm for 2 min. Add 0.3 mL chloroform and homogenise for 30 seconds and centrifuge at 12000 rpm for 2 min.
4. Add 0.3 mL water and homogenise for 0.5 min; and centrifuge at 12000 rpm for 5 min.
5. Collect the bottom layer (chloroform-lipid) by syringe with needle to a pre-weighted HPLC vial.
6. Add 0.3 mL chloroform to the centrifuge tube and homogenise for 0.5 min, and centrifuge the mixture at 12000 rpm for 5 min. Collect the bottom layer to the same HPLC vial.
7. Add 0.3 mL chloroform to the centrifuge tube and homogenise for 0.5 min, centrifuge the mixture at 12000 rpm for 5 min and collect the bottom layer to the same HPLC vial.
8. Add 0.3 mL chloroform to the centrifuge vial, homogenise for 0.5 min, centrifuge the mixture at 12000 rpm for 5 min.
9. Remove the supernatant and collect the bottom layer in the same HPLC vial.
10. Air dry the chloroform solution in the HPLC vial in a fume hood.
11. Further dry the HPLC vial at room temperature under vacuum.
12. Weigh HPLC vial and calculate the lipid amount.
13. Calculate the lipid content based on the lipid amount and biomass amount.

### Controlled Yeast Fermentation

Once a preferred C:N ratio was determined for the shake flask, the yeast with the highest intracellular lipid production was selected and subsequent cultivations were scaled up and performed in the 1-10 L TWV fermenters.

Debaryomyces hansenii CF10 was selected, and a 2-stage fed-batch fermentation process was developed in order to produce high cell density and intracellular lipids.

### 2-Stage Fed Batch Fermentation

Seed preparation:
1. Prepare and sterilize 400 mL YPD under sterilized conditions in a 1 L shake flask, as described above. Prepare 2 flasks.
2. Leave flask/s to cool to room temperature inside a biosafety cabinet.
3. Optional: When cooled down, add ampicillin, final concentration 100 ug/mL.
4. Add 0.5-1 mg of D. hansenii CF10 powder.
5. Incubate at 28° C., 200 rpm, 48 hrs.
6. QC tests:
   6.1 After 24 hrs, remove 0.05-0.1 mL from each flask and plate out onto YPD and LB agar plates (under sterilized conditions). Incubate plates at 28° C. and check for bacterial contamination.
   6.2 Check culture under microscope. Take images (if capability is available).
   6.3 At the 48-hr mark, remove flasks, and check under the microscope.
   6.4 Check plated out agar plates after 24 hrs.
7. Choose clean flask for inoculation into fermenter vessel.

### Stage 1 Fermentation

Except for sucrose, the following ingredients were added to the vessel, stirred before sterilization at 121° C. for 0.45-1 hr. Once cooled to ˜50° C., the sucrose was filter sterilized using a 0.2 μm and added into the vessel.

TABLE 1

| No | Components | Chemical name | Concentration (g/L) |
|---|---|---|---|
| 1 | Sucrose | Sucrose | 59.8 |
| 2 | Yeast extract | Yeast extract | 0.5 |
| 3 | MgSO₄7 H₂O | Magnesium sulfate heptahydrate | 1.6 |
| 4 | KH₂PO₄ | Potassium dihydrogen phosphate | 7.5 |
| 5 | Na₂HPO₄ | Sodium phosphate dibasic | 2.7 |
| 6 | (NH₄)₂SO₄ | Ammonium sulfate | 3 |
| 7 | CaCl₂•2H₂O | Calcium Chloride Dihydrate | 0.1 |
| 8 | C₂H₃NaO₂ | Sodium Acetate | 4.1 |

| Trace element solution | | | |
|---|---|---|---|
| No | Components | Chemical name | Concentration (mg/L) |
| 1 | FeCl₃•6 H₂O | Iron (III) Chloride Hexahydrate | 8 |
| 2 | ZnSO₄•7 H₂O | Zinc Sulfate Heptahydrate | 0.1 |
| 3 | CuSO₄•5 H₂O | Copper (II) sulfate pentahydrate | 0.1 |
| 4 | CoCl₂ H₂O | Cobalt (II) Chloride Hexahydrate | 0.1 |
| 5 | MnSO₄•5 H₂O | Manganese (II) sulfate pentahydrate | 0.1 |

The seed culture was inoculated into the fermenter and the run started using the following conditions.

Fermentation initial conditions:
Agitation/TIPSPD (Rushton): 78.5 cm/sec
Temperature: 28° C.
pH: Controlled not to drop below 5.5 (and do not exceed 6.5), using 3 M NaOH and 3 M HCl
Dissolved oxygen: 30%
Airflow: 1 VVM
Fermentation days: 5 days total
Fermentation working volume: 0.8 L
Antifoam: SILFAX D5100 (1:1) with MQ H₂O

Cascade conditions:
TIPSPD: 78.5-157 cm/sec
Airflow: 1 VVM
DO set at 30%

The pH was controlled not to drop below 5.5.

### Stage 2 Feed Addition

At 18 hrs elapsed fermentation time (EFT), fresh sterile 4 L medium was added to the vessel. The fermentation was continued until 120 hrs EFT.

### Ongoing Monitoring

During the run, 5-10 mL samples were removed daily and monitored for (1), contamination by dilution and plating 50-100 μL onto YPD and LB agar plates, 28-37° C., 24-48 hrs, (2) cell density by dilution and measuring 1 mL samples using a spectrophotometer set at 600 nm, and (3), microscopic imaging (40-100×).

### Downstream Processing

Harvesting: Following fermentation, the cells were collected at 10-15,000 g, 15-20 mins, 4° C. The supernatant was removed, and the wet cells were processed according to the application.

Freeze drying: Wet cells were frozen inside plastic containers at −80° C., 24 hrs. Frozen cells were freeze-dried at −85° C., under vacuum for 48-72 hrs.

Spray drying: Cells (e.g. 266 g in 800 mL water) were spray dried, using the following conditions; Inlet temperature 160° C., Outlet temperature 70° C., and Pump rate 40%.

Cell lysis spray drying: Cells were also lysed at cells to liquid ratio (1:3) under 40 Kpsi, 2-3 passes, before spray drying. The spray drying conditions were, Inlet temperature 160° C., Outlet temperature 70° C., and Pump rate 40%.

### Dairy-Like Compositions

Dairy-like compositions were formulated as set out in Table 2. Extraction of total oil/lipids from the yeasts was performed using the modified Bligh and Dyer chloroform/methanol solvents as described in the materials and methods.

TABLE 2

| | | | | | Yeast | Yeast | Yeast | Sunflower | Skim | Rennet | Plant |
|---|---|---|---|---|---|---|---|---|---|---|---|
| | AS1 | BCn | AS2 | Kappa | Oil* | Whole* | lysed* | Oil | milk | casein | protein |
| Composition | % | % | % | % | % | % | % | % | % | % | % |
| No | (w/v) | (w/v) | (w/v) | (w/v) | (w/v) | (w/v) | (w/v) | (w/v) | (w/v) | (w/v) | (w/v) |
| 1 | 6 | 6 | | | 1.4 | | | | | | |
| 2 | 6 | 6 | | | | 7 | | | | | |
| 3 | 6 | 6 | | | | 7 | | | | | |
| 4 | | | | | | | | 10 | 24 | | |
| 5 | | | | | | | | 20 | 24 | | |
| 6 | | | | | | | | 14 | 12 | | |
| 7 | 6 | 6 | | | | 3.5 | | | | | |
| 8 | 12 | | | | | | 3.5 | | | | |
| 9 | 12 | | | | | | 3.5 | 10.5 | | | |
| 10 | 6 | 6 | | | | | | 14 | | | |
| 11 | 6 | | | | | | 3.6 | 10.5 | | | 6 |
| 12 | | | | | | | | 14 | | | 12 |
| 13 | | | | | | | | 14 | | 12 | |

Components were mixed with water and heated at 75° C., 15 mins
Composition 3, included 0.6% CaCl₂, and 0.36% citric acid
Composition 6, included 0.006% citric acid
*Debaryomyces hansenii CF10

### Results

### Batch Fermentation

Terrific broth medium was optimized to use glycerol at 1%, not 0.5%, and IPTG was added at a concentration of 0.2 mM, not 1 mM. These amounts were preferred for controlled fermentations up to 10 L TWV. Furthermore, timing for addition of IPTG was determined to be 9 at OD600 nm.

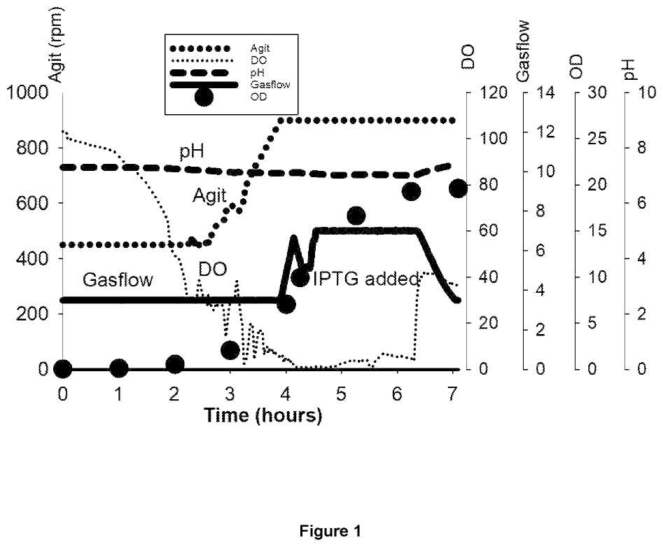

A typical trend of a recombinant E. coli-casein fermentation batch run is illustrated in FIG. 1. As indicated above IPTG was added at OD of 9, and stopped at 3 hrs post IPTG addition.

### Downstream Processing

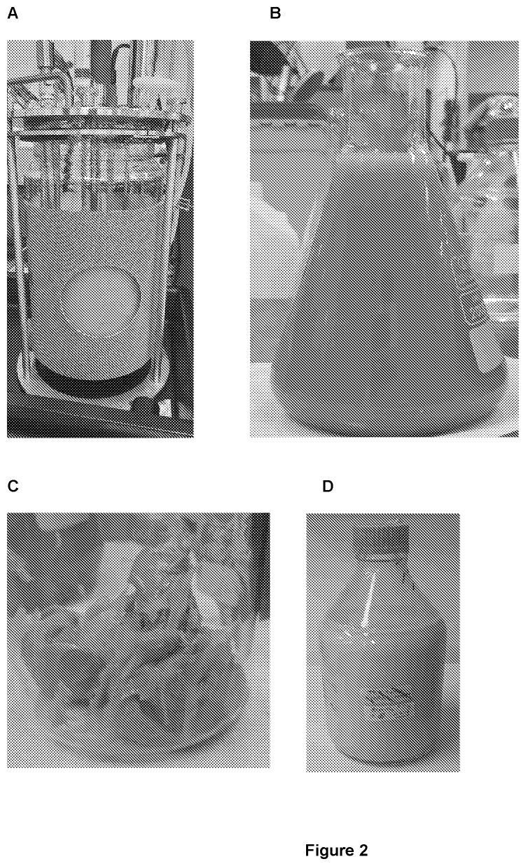

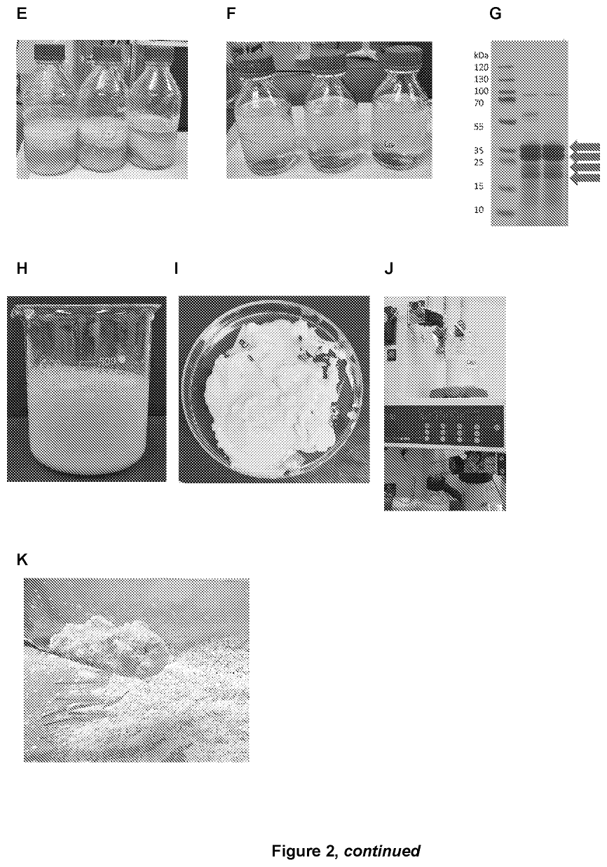

Following harvesting, typical yields of wet E. coli cell biomass were measured on average at ˜40 g/L w/v (and at about ˜60 g/L when cells were grown at room temperature). Cells were lysed and processed through a 2-stage purification in order to isolate the casein proteins from E. coli cell debris and host proteins (FIG. 2).

As shown in FIG. 2, recombinant caseins went through a 2-stage purification process by (1), heat treatment at 70-80° C., 1-2 hrs, and (2), pl/isoelectric point precipitation. Isoelectric point precipitation was dependent on the theoretical protein pl. Casein curd-like aggregates precipitated out of solution and were washed in sterile water, and spray dried. Heat treatment at other temperatures and for different lengths of time was also found to be effective.

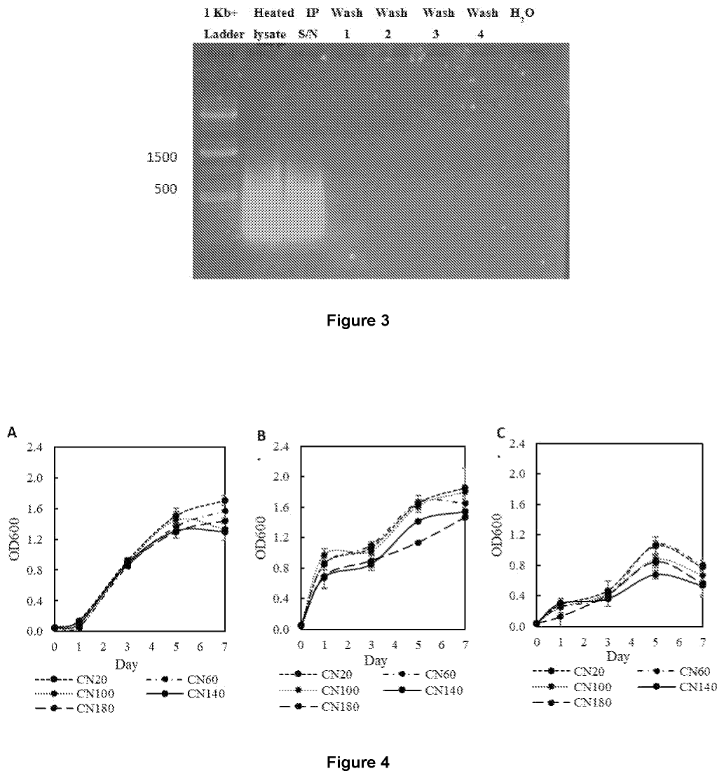

Casein aggregates were re-solubilized using strong denaturants and visualised using SDS-PAGE. High purity of up to 95% of casein proteins was achieved. Agarose gel electrophoresis demonstrated that host DNA was removed after the 2nd water wash post isoelectric point precipitation (FIG. 3).

### Yeast Lipid Production

The three yeast strains, Debaryomyces hansensii (D. hansenii CF10), Yarrowia lipolytica (Y. lipolytica CF4) and Klyveromyces lactis (K. lactis CF3) were chosen for lipid production. All three strains are of food-grade, and used in traditional cheese making.

The three yeasts were first tested under different C:N ratios, and their growth was monitored over time, at 25° C. (FIG. 4). D. hansenii (A) and K. lactis (B) had similar OD600 (cell density) values by day 7, under the C:N 20 ratios. Y. lipolytica (C) peaked by Day 5 with similar OD600 values at the C:N 20 and 60 ratios. However, the lower values for Y. lipolytica may be due to the growth temperature of 25° C.

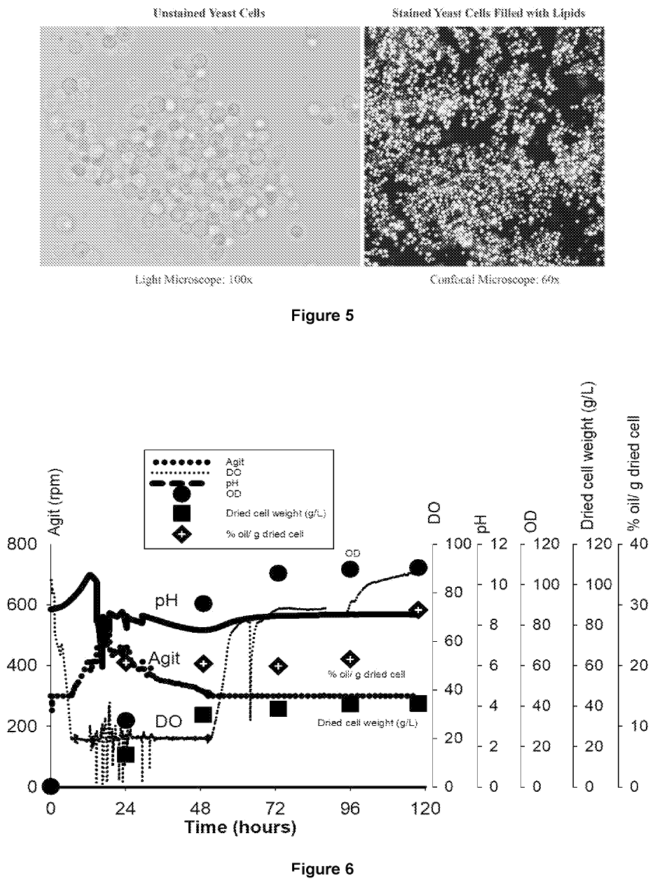

Cells were also stained with Nile Red to detect intracellular buildup of lipids, under fluorescence (FIG. 5). As an example, FIG. 5 presents microscopy images of D. hansenii CF 10. Unstained (Left) and Stained (Right).

After testing different variables, preferred conditions included the use of sucrose as carbon source and addition of sodium acetate in a fed-batch process, producing high yeast cell density of >100 and total oils/lipids at 12 g/L (FIG. 6). High cell density and total lipids was achieved in a shorter timeframe of 5 days down from 7 days.

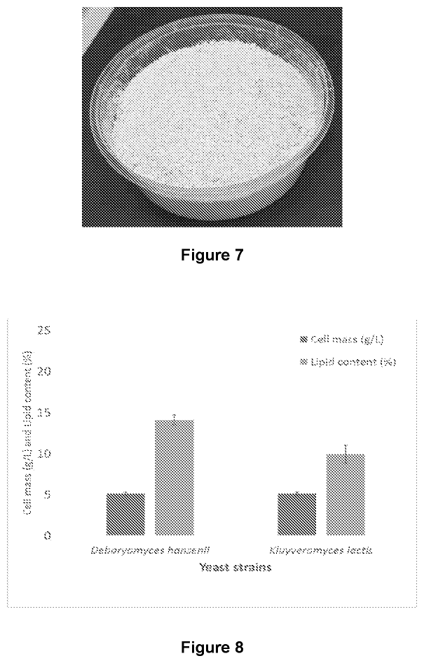

Following fermentation and harvesting, whole cells were spray dried resulting in a light pink milky white fine powder with a moisture content of ˜5-8% (FIG. 7).

A comparison of total lipids between D. hansenii and K. kactis is shown in FIG. 8, with yields of ˜15% (w/w) and 10% (w/w), respectively. A comparison of the main fatty acid composition of different yeasts with cow's milk, cheddar and mozzarella cheese as a percentage by weight of total fatty acids is shown in Table 3. Cow's milk, for example, contains about 4.1 grams of fat in 100 grams of milk. Of that 4.1 grams, 70% is saturated fatty acids (SFA) and 27.3% is unsaturated fatty acids (UFA), and of the 4.1 grams, 10.9% is myristic acid. Overall, the profiles of the D. hansenii and K. lactis are comparable to plain cow's milk, and to the more developed cheese products cheddar and mozzarella. In general, higher amounts of total unsaturated fatty acids were present in D. hansenii and K. lactis.

TABLE 3

| | | | | | | | | | Total | |
|---|---|---|---|---|---|---|---|---|---|---|
| | C14:0 | C16:0 | C18:0 | C18:1 | C18:2 | C18:3 | | | Fats % | |
| Source | myristic | palmitic | stearic | oleic | linoleic | linolenic | SFA | UFA | (g/100 g) | Reference |
| Cow's milk | 10.9 | 30.6 | 12.2 | 22.8 | 1.6 | 0.5 | 70 | 27.3 | 4.1 | (Lindmark |
| | | | | | | | | | | Mansson, 2008) |
| Cheddar | 10.6 | 28.9 | 10.9 | 20.6 | 1.8 | T | 66.7 | 33.3 | 39.8 | (Manuelian et |
| | | | | | | | | | | al., 2017) |
| Mozzarella | 10.7 | 31.1 | 9.9 | 20.6 | 2.2 | T | 67.7 | 32.3 | 17.7 | (Manuelian et |
| | | | | | | | | | | al., 2017) |
| D. hansenii | T | 18.9 | 10.5 | 43.7 | 18.7 | 4.7 | 29.4 | 67.1 | 7.0^{d} | (Kaneko et al., |
| MY-45^{a} | | | | | | | | | | 1976) |
| Y. lipolytica | T | 17.8 | 8.3 | 35.7 | 29.3 | T | 26.7 | 73.3 | 10^{d} | (Andre et al., |
| LFMB 19^{b} | | | | | | | | | | 2009) |
| K. lactis | T | 9.4 | 2.1 | 23.2 | 34.7 | 6.5 | 12.4 | 87.6 | 5.9^{d} | (Tanji et al., |
| NBRC 1090^{c} | | | | | | | | | | 2004) |
| D. hansenii | 0.3 | 28.2 | 11.5 | 32.0 | 8.0 | 0.9 | 51.7 | 48.3 | 12.3 | This work |
| CF10^{b} | | | | | | | | | | |
| K. lactis CF3^{b} | 0.4 | 24.2 | 14.0 | 21.0 | 9.9 | 0.7 | 61.7 | 38.3 | 6.7 | This work |

T: trace,
SFA: saturated fatty acid,
UFA: unsaturated fatty acid,
FA: fatty acids,
^{a}in YM-medium at 30° C.,
^{b}in nitrogen-limited media containing crude glycerol at early stationary ((150-170 h) growth phase at 28° C.,
^{c}in YPD medium at 25° C.,
^{d}Expressed as total lipids in g/100 g dry cell weight.

### Dairy-Like Compositions

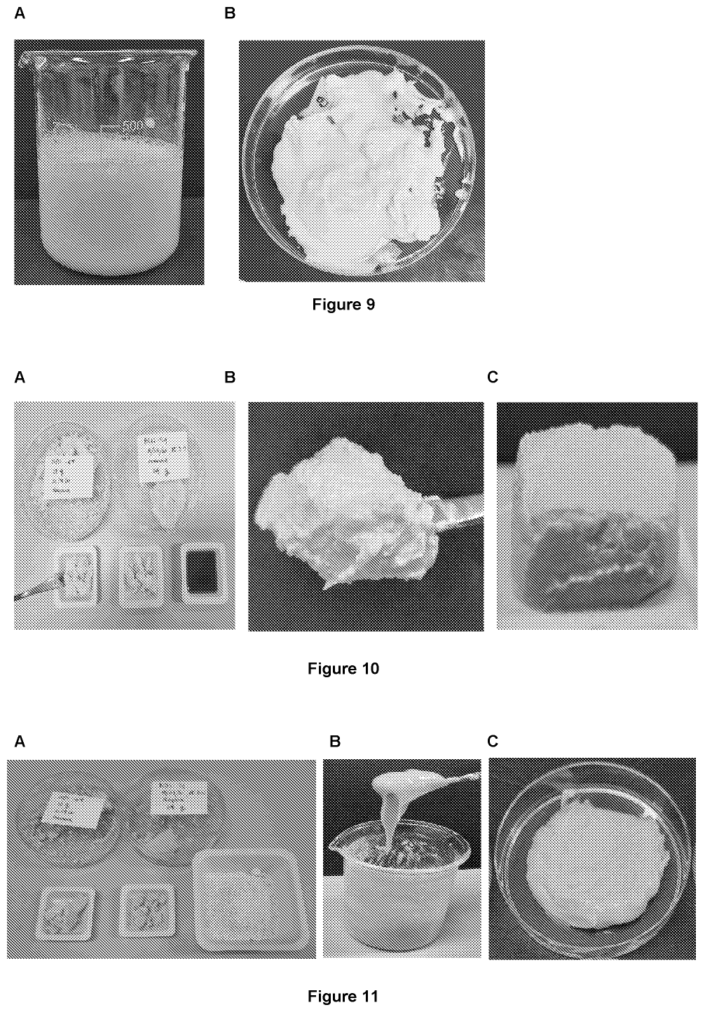

Purified recombinant caseins formed a curd-like coagulation structure even at room temperature (RT) as shown in FIG. 9. This type of casein protein aggregation was typical of all recombinant caseins produced following the 2-stage purification process.

Demonstration of two recombinant caseins and oil isolated from yeast in a dairy-like composition is shown in FIG. 10. All the components were mixed together at 75° C., 15 mins, with occasional stirring. Samples held the curd-like matrix structure at RT and 4° C.

Demonstration of two recombinant caseins and whole yeast aggregation in a dairy-like composition is shown in FIG. 11. All the components were mixed together at 75° C., 15 mins, with occasional stirring. Samples held the curd-like matrix structure at RT and 4° C.

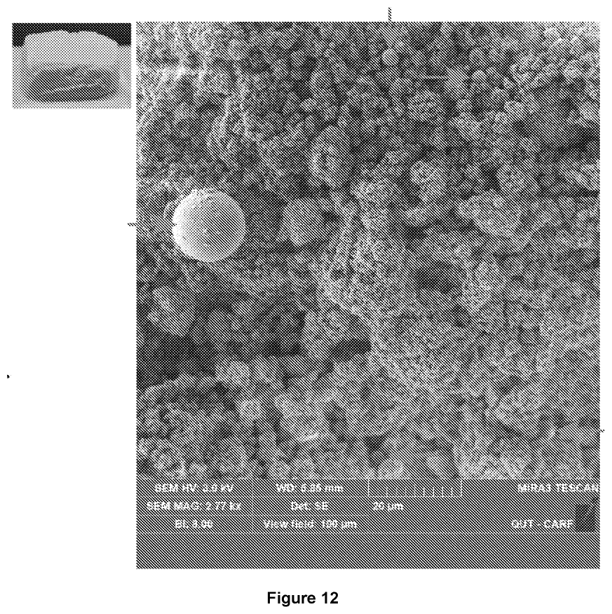

FIG. 12 presents a scanning electron microscope (SEM) image (20 μm) of a dairy-like composition of alpha S1, beta casein proteins and yeast oil. Arrows point to visible yeast oil droplets amongst aggregated proteins. The recombinant casein proteins formed a close aggregation together with the yeast oil droplets.

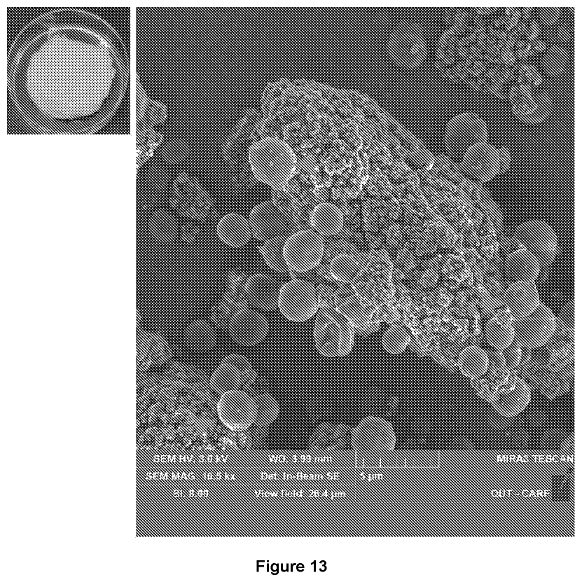

FIG. 13 presents an SEM image (5 μm) of a dairy-like composition of alpha S1, beta casein proteins and whole yeast cells. Arrows point to whole yeast cells containing intracellular lipids amongst aggregated proteins. The recombinant casein proteins formed a close aggregation together with the yeast cells containing lipids.

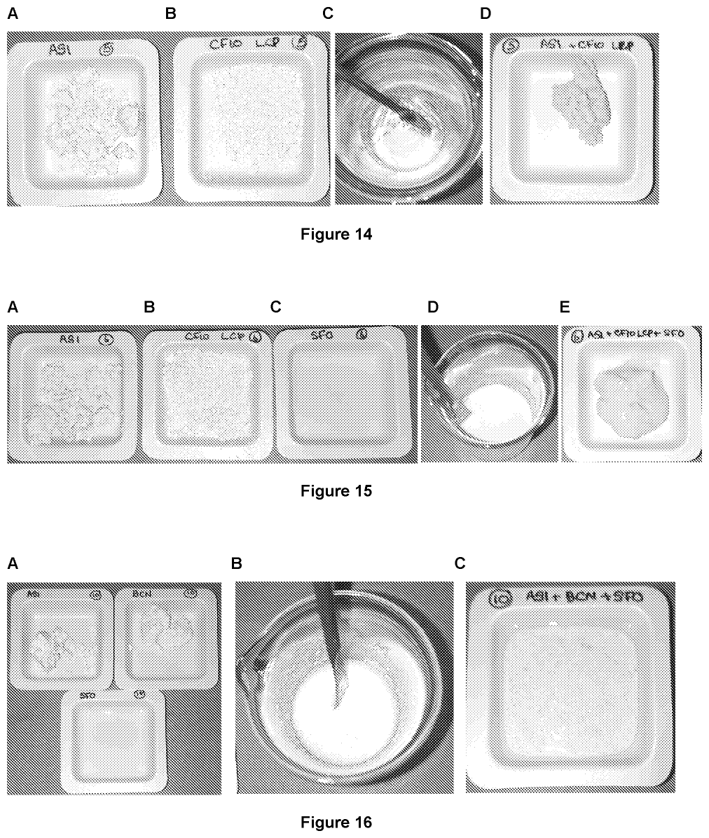

Demonstration of recombinant alpha-S1 casein and lysed spray dried yeast in a dairy-like composition is shown in FIG. 14. All components were mixed together at 75° C., 15 mins, with occasional stirring. Samples held the curd-like matrix structure at RT and 4° C.

Demonstration of recombinant alpha-S1 casein, lysed spray dried yeast and sunflower oil in a dairy-like composition is shown in FIG. 15. All components were mixed together at 75° C., 15 mins, with occasional stirring. Samples held the curd-like matrix structure at RT and 4° C.

Demonstration of recombinant alpha-S1 casein, recombinant beta casein and sunflower oil in a dairy-like composition is shown in FIG. 16. All components were mixed together at 75° C., 15 mins, with occasional stirring. Samples held the curd-like matrix structure at RT and 4° C.

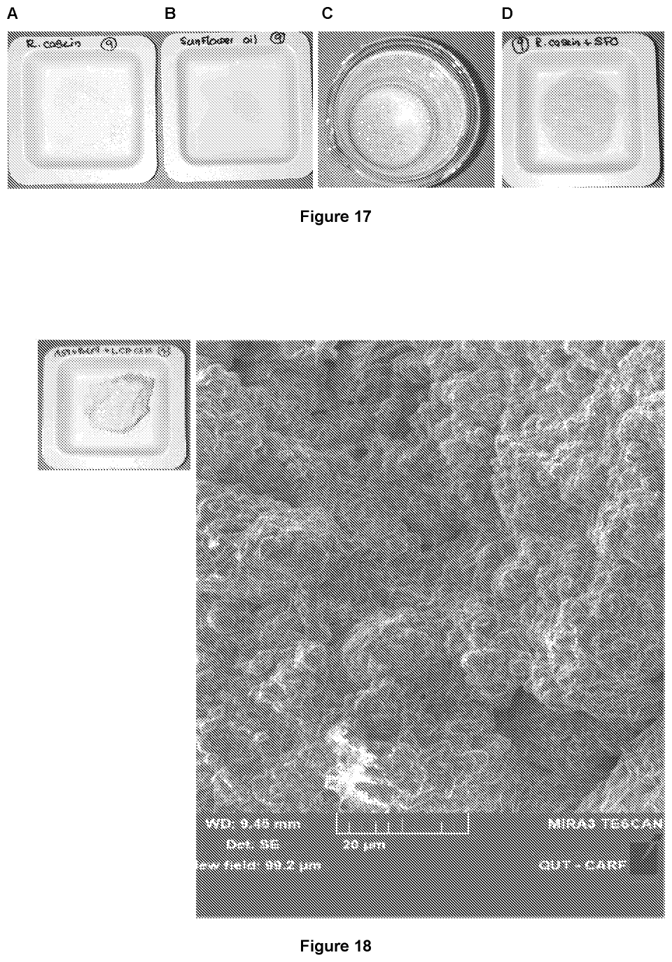

Demonstration of rennet casein and sunflower oil in a dairy-like composition is shown in FIG. 17. All the components were mixed together at 75° C., 15 mins, with occasional stirring.

FIG. 18 presents an SEM image (20 μm) of a dairy-like composition of recombinant alpha S1 casein, recombinant beta casein and lysed spray dried yeast.

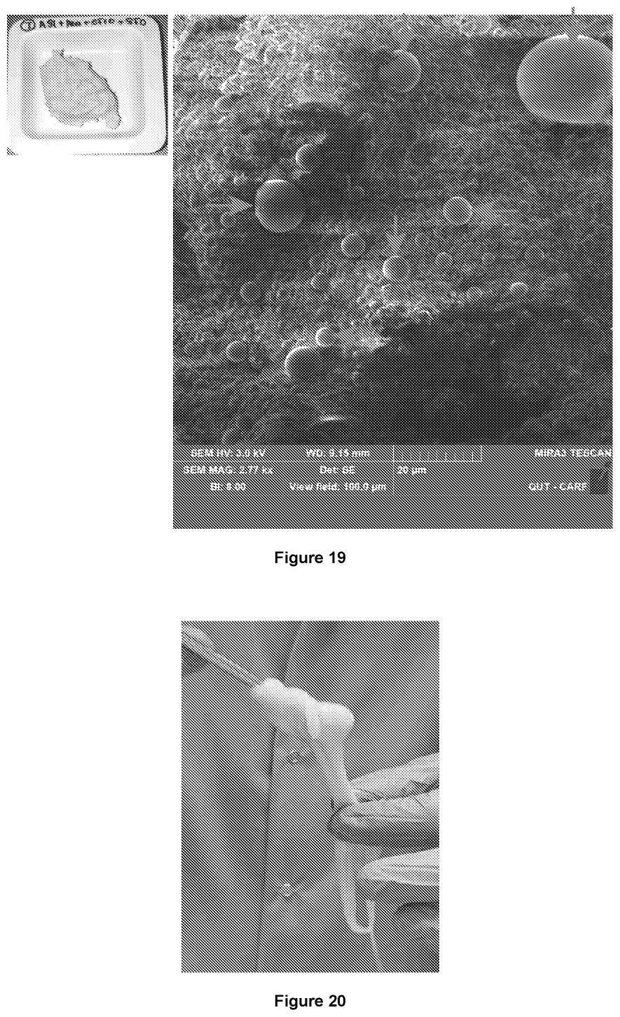

FIG. 19 presents an SEM image (20 μm) of a dairy-like composition of recombinant alpha S1 casein, pea protein, sunflower oil and lysed spray dried yeast. Arrows point to visible oil droplets amongst aggregated proteins.

### Cheese Composition

A cheese composition was produced by mixing the ingredients listed in Table 4 using an RVA 4800 cooking capsule (Perkin Elmer).

TABLE 4

| Ingredient | Weight (g) |
|---|---|
| Water | 14.5 |
| Fat (AkoVeg ™ 107-12, Coconut Oil/Sunflower Oil) | 7.0 |
| Alpha S1 recombinant casein | 6.45 |
| Sodium Aluminum Phosphate Basic - Food Grade | 0.65 |
| Starch | 1.2 |

The ingredients were mixed under the conditions listed in Table 5.

TABLE 5

| Stage | Temperature (° C.) | Mixing Speed (rpm) | Time (mins) |
|---|---|---|---|
| I | Ramping, 0-70 | 100 | 2 |
| II | 70 | 300 | 9 |

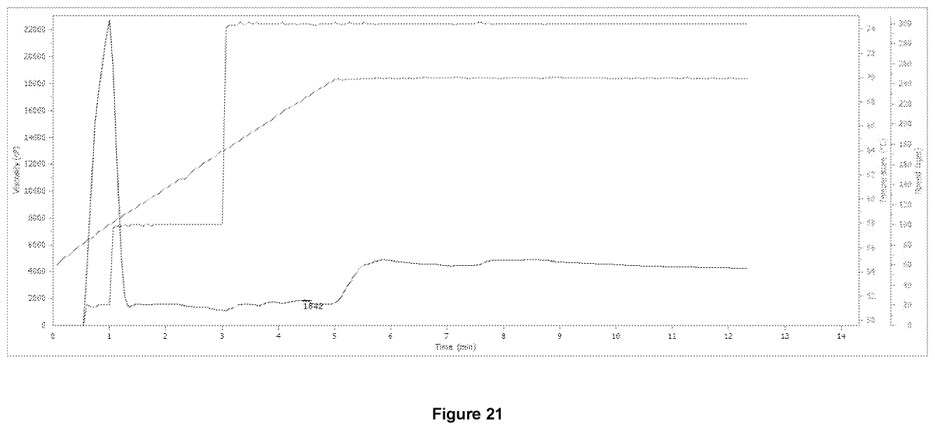

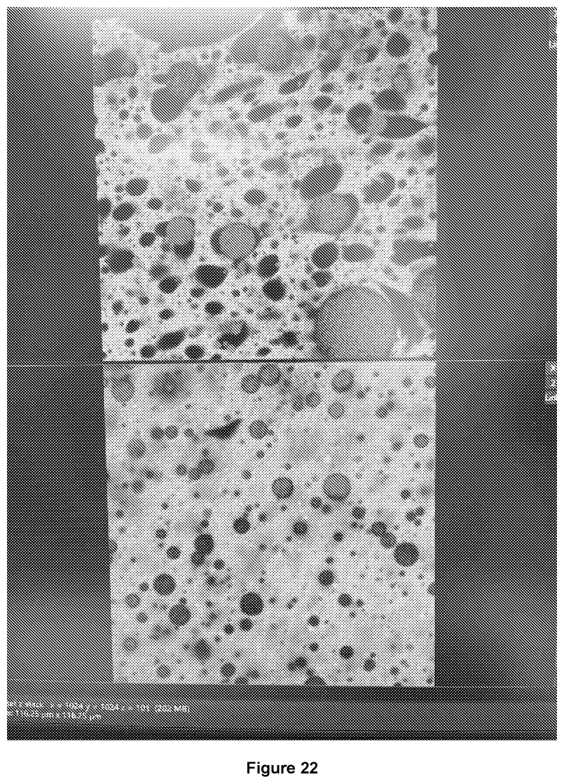

After cooking and cooling, the composition comprising recombinant alpha S1 had the texture, colour and aroma of dairy cheese (FIG. 20). The viscosity (cP), temperature (° C.) and agitation speed (rpm) during the recombinant alpha S1 cheese manufacturing process are shown in FIG. 21. Confocal imaging of the recombinant alpha S1 cheese composition revealed the presence of lipid droplets dispersed throughout the protein (FIG. 22).

It will be appreciated by those skilled in the art that the present disclosure may be embodied in many other forms.

1. A dairy-like composition comprising:
one or more recombinant casein proteins or fragments thereof; and
microbial lipids.

2. The composition of claim 1 wherein the composition is milk-free.

3. The composition of claim 1 or claim 2 wherein the one or more recombinant casein proteins are selected from the group consisting of αS1-casein, αS2-casein, β-casein and κ-casein. 4 The composition of any one of claims 1 to 3 wherein the one or more casein proteins comprise αS1-casein and/or β-casein.

5. The composition of any one of claims 1 to 4 wherein at least one of the recombinant casein proteins is expressed in a microorganism from a codon optimised gene.

6. The composition of any one of claims 1 to 5 wherein the one or more casein proteins are produced by a microorganism.

7. The composition of any one of claims 1 to 6 wherein the one or more casein proteins and the microbial lipids are produced by different microorganisms.

8. The composition of any one of claims 1 to 7 wherein the one or more casein proteins and the microbial lipids are produced by different species of microorganisms

9. The composition of any one of claims 1 to 8 wherein the one or more recombinant casein proteins are isolated from E. coli or a species of Trichoderma.

10. The composition of any one of claims 1 to 9 wherein at least one of the recombinant casein proteins is isolated from a microorganism by a process comprising:
i) lysing the microorganism to produce a cell lysate;
ii) heating the cell lysate to produce a heat-treated lysate;
iii) centrifuging the heat-treated lysate and obtaining a supernatant;
iv) adding an acid to the supernatant so as to lower its pH and promote precipitation of the casein protein; and
v) centrifuging the supernatant to form a pellet of casein protein.

11. The composition of claim 10 wherein the process further comprises:
vi) resuspending the pellet of casein protein in an aqueous solution; and
vii) freeze drying or spray drying the resuspended casein protein.

12. The composition of any one of claims 1 to 11 wherein the microbial lipids are produced by a microorganism that is not recombinant.

13. The composition of any one of claims 1 to 12 wherein the microbial lipids are yeast lipids.

14. The composition of claim 13 wherein the yeast is Debaryomyces hansenii, Kluyveromyces lactis or Yarrowia lipolytica.

15. The composition of claim 13 wherein the yeast is Debaryomyces hansenii.

16. The composition of any one of claims 13 to 15 wherein the composition comprises whole yeast cells, and wherein the yeast cells comprise the lipids.

17. The composition of claim 16 wherein the yeast cells are obtained by spray drying or freeze drying.

18. The composition of any one of claims 13 to 17 wherein the composition comprises lysed spray dried yeast cells, and wherein the yeast cells comprise the lipids.

19. The composition of any one of claims 13 to 18 wherein the composition comprises lipids extracted from yeast cells.

20. The composition of any one of claims 1 to 19 wherein the composition comprises recombinant casein proteins at a concentration of between 5% and 25%.

21. The composition of any one of claims 1 to 20 wherein the composition comprises microbial lipids at a concentration of between 1% and 20%.

22. The composition of any one of claims 1 to 21 wherein the composition comprises microbial lipids at a concentration of between 1% and 5%.

23. The composition of any one of claims 1 to 22 wherein the microbial lipids comprise palmitic acid, stearic acid, oleic acid, linoleic acid and linolenic acid.

24. The composition of any one of claims 1 to 23 wherein the composition further comprises a plant oil or a plant protein.

25. The composition of any one of claims 1 to 24 wherein the composition comprises between about 4% and 40% lipids, and wherein a fraction of the lipids are microbial lipids.

26. The composition of claim 25 comprising between about 1% and 5% microbial lipids and between about 5% and 40% total lipids.

27. A method of producing a dairy-like composition the method comprising mixing one or more recombinant casein proteins or fragments thereof with microbial lipids.

28. The method of claim 27 wherein the composition is milk-free.

29. The method of claim 27 or claim 28 wherein the mixing is performed at a temperature of between about 70° C. and 80° C.

30. The method of any one of claims 27 to 29 wherein the microbial lipids are yeast lipids.

31. The method of claim 30 wherein the yeast is Debaryomyces hansenii, Kluyveromyces lactis or Yarrowia lipolytica.

32. The method of claim 30 or claim 31 wherein the lipids are added to the composition in the form of whole yeast cells comprising the lipids.

33. The method of any one of claims 30 to 32 wherein the lipids are added to the composition in the form of lysed yeast cells comprising the lipids.

34. The method of claim 32 or claim 33 wherein the yeast cells are prepared by spray drying or freeze drying.

35. The method of any one of claims 30 to 34 wherein the lipids are extracted from yeast cells.

36. The method of any one of claims 30 to 35 wherein the one or more recombinant casen proteins are isolated from bacteria such as E. coli or a species of Trichoderma.

37. A food or beverage product comprising a dairy-like composition of any one of claims 1 to 26.


SEQUENCE LISTING (18 sequences)

SEQ ID NO: 1 - protein, 214 aa, synthetic construct
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 199 aa, synthetic construct
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 222 aa, synthetic construct
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 4 - protein, 207 aa, synthetic construct
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 5 - protein, 224 aa, synthetic construct
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 6 - protein, 209 aa, synthetic construct
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 7 - DNA, 645 bp, synthetic construct
  source 1..645: mol_type = other DNA
atgaagctgctgatcctgacctgcctggtggccgtggccctggcccgtccgaagcatccgattaagcaccagggtctgccgcaagaggttctgaacgaaaacctgctgcgtttctttgtggcgccgttcccggaagtgttcggcaaggagaaagtgaacgaactgagcaaggacatcggcagcgagagcaccgaggaccaggcgatggaggatattaaacaaatggaggcggagagcatcagcagcagcgaggaaattgtgccgaacagcgttgaacagaagcacatccaaaaagaggatgtgccgagcgaacgttacctgggctatctggagcagctgctgcgtctgaagaaatacaaggttccgcaactggaaatcgttccgaacagcgcggaggaacgtctgcacagcatgaaagagggtatccacgcgcagcaaaaagaaccgatgattggcgttaaccaggagctggcgtacttctatccggaactgttccgtcagttttaccaactggacgcgtatccgagcggtgcgtggtactatgtgccgctgggcacccaatataccgatgcgccgagctttagcgacattccgaacccgattggcagcgaaaatagcgaaaaaaccaccatgccgctgtggtaa

SEQ ID NO: 8 - DNA, 669 bp, synthetic construct
  source 1..669: mol_type = other DNA
atgaagttcttcatcttcacctgcctgctggccgtggccctggccaagaacaccatggagcacgtgagcagcagcgaggagagcatcatcagccaggagacctacaagcaggagaagaacatggccatcaaccccagcaaggagaacctgtgcagcaccttctgcaaggaggtggtgaggaacgccaacgaggaggagtacagcatcggcagcagcagcgaggagagcgccgaggtggccaccgaggaggtgaagatcaccgtggacgacaagcactaccagaaggccctgaacgagatcaaccagttctaccagaagttcccccagtacctgcagtacctgtaccagggccccatcgtgctgaacccctgggaccaggtgaagaggaacgccgtgcccatcacccccaccctgaacagggagcagctgagcaccagcgaggagaacagcaagaagaccgtggacatggagagcaccgaggtgttcaccaagaagaccaagctgaccgaggaggagaagaacaggctgaacttcctgaagaagatcagccagaggtaccagaagttcgccctgccccagtacctgaagaccgtgtaccagcaccagaaggccatgaagccctggatccagcccaagaccaaggtgatcccctacgtgaggtacctgtag

SEQ ID NO: 9 - DNA, 675 bp, synthetic construct
  source 1..675: mol_type = other DNA
atgaaggtgctgatcctggcctgcctggtggccctggccctggcccgtgagctggaagaactgaacgtgccgggtgaaatcgttgagagcctgagcagcagcgaggaaagcatcacccgtatcaacaagaaaattgagaagttccaaagcgaggaacagcaacagaccgaggacgagctgcaagataaaatccacccgttcgcgcaaacccagagcctggtgtacccgtttccgggtccgatcccgaacagcctgccgcaaaacattccgccgctgacccagaccccggtggttgtgccgccgtttctgcaaccggaagtgatgggcgtgagcaaggttaaagaggcgatggcgccgaagcacaaagaaatgccgttcccgaagtatccggtggagccgtttaccgaaagccaaagcctgaccctgaccgacgttgaaaacctgcacctgccgctgccgctgctgcaaagctggatgcatcaaccgcaccaaccgctgccgccgaccgtgatgttcccgccgcaaagcgttctgagcctgagccagagcaaggtgctgccggttccgcagaaagcggttccgtacccgcaacgtgatatgccgattcaggcgtttctgctgtatcaagagccggttctgggtccggtgcgtggtccgtttccgattattgtgtaa

SEQ ID NO: 10 - protein, 190 aa, synthetic construct
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 11 - protein, 169 aa, synthetic construct
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 12 - DNA, 573 bp, synthetic construct
  source 1..573: mol_type = other DNA
atgatgaaatcatttttcctagtagttacaatcctggcactgaccctgccgtttctgggtgctcaagagcagaaccaagagcaaccgatccgctgcgaaaaagatgaacgcttcttcagcgataagattgccaagtacatcccgatccagtatgtgttgtctcgttatccgagctatggcctcaattattaccagcagaagccggttgctttgatcaataaccaatttttgccctacccgtactacgcaaaaccggcggcggttcgtagcccggcacagattcttcaatggcaggttctgtccaataccgttccggcgaaaagctgtcaggcgcagccgaccaccatggcgcgtcacccgcatccgcacctgtcgttcatggcgattccgcctaaaaagaaccaagacaagaccgagattccgacgattaacaccatcgcgagcggtgaaccgacttctacgccgaccaccgaggcggtcgaaagcacggtggctaccctggaagactccccagaggtaattgaatccccgccagagatcaacaccgtgcaggtgacctcaactgccgtctaa

SEQ ID NO: 13 - protein, 210 aa, synthetic construct
MRELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 14 - protein, 209 aa, synthetic construct; identical to SEQ ID NO: 6

SEQ ID NO: 15 - DNA, 633 bp, synthetic construct
  source 1..633: mol_type = other DNA
atgcgcgaactggaagaactgaacgtgccgggcgaaattgtggaaagcctgagcagcagcgaagaaagcattacccgcattaacaaaaaaattgaaaaatttcagagcgaagaacagcagcagaccgaagatgaactgcaggataaaattcatccgtttgcgcagacccagagcctggtgtatccgtttccgggcccgattccgaacagcctgccgcagaacattccgccgctgacccagaccccggtggtggtgccgccgtttctgcagccggaagtgatgggcgtgagcaaagtgaaagaagcgatggcgccgaaacataaagaaatgccgtttccgaaatatccggtggaaccgtttaccgaaagccagagcctgaccctgaccgatgtggaaaacctgcatctgccgctgccgctgctgcagagctggatgcatcagccgcatcagccgctgccgccgaccgtgatgtttccgccgcagagcgtgctgagcctgagccagagcaaagtgctgccggtgccgcagaaagcggtgccgtatccgcagcgcgatatgccgattcaggcgtttctgctgtatcaggaaccggtgctgggcccggtgcgcggcccgtttccgattattgtgtaa

SEQ ID NO: 16 - protein, 182 aa, synthetic construct
MKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 17 - protein, 181 aa, synthetic construct
KIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 18 - DNA, 546 bp, synthetic construct
  source 1..546: mol_type = other DNA
atgaagatcgagaagttccagagcgaggagcagcagcagaccgaggacgagctgcaggacaagatccaccccttcgcccagacccagagcctggtgtaccccttccccggccccatccccaacagcctgccccagaacatcccccccctgacccagacccccgtggtggtgccccccttcctgcagcccgaggtgatgggcgtgagcaaggtgaaggaggccatggcccccaagcacaaggagatgcccttccccaagtaccccgtggagcccttcaccgagagccagagcctgaccctgaccgacgtggagaacctgcacctgcccctgcccctgctgcagagctggatgcaccagccccaccagcccctgccccccaccgtgatgttccccccccagagcgtgctgagcctgagccagagcaaggtgctgcccgtgccccagaaggccgtgccctacccccagagagacatgcccatccaggccttcctgctgtaccaggagcccgtgctgggccccgtgagaggccccttccccatcatcgtg